SEQUENCE LISTING (12 sequences)

SEQ ID NO: 1 - protein, 1529 aa, Homo sapiens
MRGVGWQMLSLSLGLVLAILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRTSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGCTRCVS

SEQ ID NO: 2 - protein, 1119 aa, Homo sapiens
MRGVGWQMLSLSLGLVLAILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLP

SEQ ID NO: 3 - protein, 234 aa, Homo sapiens
LHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADL

SEQ ID NO: 4 - protein, 1093 aa, Homo sapiens
QACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSDEEEGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRT

SEQ ID NO: 5 - protein, 1507 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSDEEEGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRTSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGCTRCVS

SEQ ID NO: 6 - protein, 1501 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSDEEEGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSRTSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGCTRCVS

SEQ ID NO: 7 - protein, 240 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCS

SEQ ID NO: 8 - protein, 465 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSDEEEGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCS

SEQ ID NO: 9 - protein, 692 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSDEEEGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDD

SEQ ID NO: 10 - protein, 886 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSDEEEGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQ

SEQ ID NO: 11 - protein, 1092 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSDEEEGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFS

SEQ ID NO: 12 - protein, 1165 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSDEEEGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRTSPCDNFDCQNGAQCIVRINEPICQCLPGYQGEKCEKLVSVNFINKESYLQIPSAKVRPQTNITLQ

(19) United States
(12) Patent Application Publication
(10) Pub. No.: US 2012/0129757 A1
(43) Pub. Date: May 24, 2012

(54) COMPOSITIONS AND METHODS FOR PROMOTING VASCULAR BARRIER FUNCTION AND TREATING PULONARY FIBROSIS

(76) Inventors: Dean Li, Salt Lake City, UT (US); Nyall London, Bountiful, UT (US); Weiquan Zhu, Salt Lake City, UT (US)

(21) Appl. No.: 13/000,802

(22) PCT Filed: Dec. 11, 2009
(86) PCT No.: PCT/US2009/067746

§ 371 Date: Jan. 6, 2012

Related U.S. Application Data
(60) Provisional application No. 61/122,265, filed on Dec. 12, 2008.

(51) Int. Cl. A61K 31/4196; A61P 9/00; A61K 38/17

(52) U.S. Cl. 514/1.1; 514/384

(57) ABSTRACT

Active agents and compositions that promote barrier function or that inhibit permeability of the vascular endothelium associated with pulmonary inflammation are described.

### STATEMENT REGARDING FEDERALLY SPONSORED RESEARCH

This invention was made with Government support under Grant R01 HL077671 awarded by the National Institutes of Health. The Government has certain rights to this invention.

### BACKGROUND

Acute and chronic pulmonary vascular inflammation and leak are associated with multiple pathologic conditions. For example, influenza infections and sepsis can be characterized by acute, and potentially life-threatening, pulmonary vascular inflammation. Additionally, chronic pulmonary vascular inflammation is associated with the development and progression of pulmonary fibrosis. Pulmonary fibrosis is the abnormal formation of fiber-like scar tissue in the lungs, with the scar formation being preceded by, and associated with, inflammation. Pulmonary fibrosis is a chronic disease causing swelling and scarring of the alveoli and interstitial tissues of the lungs. The cause of pulmonary fibrosis is often never determined (i.e., idiopathic pulmonary fibrosis), but in some instances, the development and progression pulmonary fibrosis is associated with a disease or infection, such as, for example, tuberculosis, systemic Lupus Erythematosis, systemic sclerosis, one or more environmental conditions, such as, for example, exposure to silica dust and asbestos, or even particular drugs, such as nitiofurantoin, amiodarone, and bleomycin. Though pulmonary fibrosis can be so mild as to cause few symptoms, it can also be fatal.

### SUMMARY OF THE INVENTION

Active agents and compositions that promote vascular barrier function are described herein. Compositions described herein include at least one active agent capable of promoting vascular barrier function, and in one such embodiment, the compositions described herein include an active agent that promotes the barrier function of vascular endothelium. In another embodiment, the compositions described herein include an active agent that promotes vascular barrier function in endothelial tissue selected from one of vascular endothelium of the lung, vascular endothelium of the kidney and vascular endothelium of the spleen. In another embodiment, a composition as described herein includes an active agent that inhibits vascular permeability associated with pulmonary inflammation, including vascular permeability associated with conditions leading to or resulting from acute pulmonary inflammation and chronic pulmonary inflammation. As illustrated by the experimental examples provided herein, active agents according to the present description, in particular embodiments, promote vascular barrier function even in the presence of multiple mediators of inflammation and vascular permeability, including, for example, endotoxins (e.g., LPS), tumor necrosis factor (e.g., TNF-α), and IL-1β.

Methods for promoting vascular endothelial barrier function are also provided herein. In one embodiment, a method for promoting vascular endothelial barrier function includes treating one or more vascular endothelial cells with an active agent as described herein. In one such embodiment, the step of treating one or more vascular endothelial cells may be carried out by administering to a patient in need thereof a therapeutically effective amount of an active agent as described herein. In particular embodiments, treatment of the one or more vascular endothelial cells with the active agent results in one or more of the following: preservation of vascular endothelial barrier function; promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cell. In another such embodiment, treatment of the one or more vascular endothelial cells with the active agent restores, at least in part, vascular barrier function after exposure of the vascular endothelial cells to one or more mediators of inflammation, wherein the one or more mediators of inflammation are selected from including one or more of an endotoxin (e.g., LPS), tumor necrosis factor (e.g., TNF-α), and IL-1β.

### BRIEF DESCRIPTION OF THE DRAWINGS

FIG. 1. Slit2N stabilizes the endothelium by enhancing VE-cadherin localization at the cell surface. (A), In vitro permeability was measured in HMVEC-lung stimulated with LPS, TNF-α, or IL-1β in the presence of Mock or Slit2N. (B), Robo4 or control siRNA knockdown HMVEC-lung were stimulated with IL-1β in the presence of Mock or Slit2N to assess permeability in vitro. (C-E), HMVEC-lung were treated with Mock or Slit2N, subjected to membrane fractionation and subsequent immunoblotting for VE-cadherin (C), p120-catenin (D), or β-catenin (E). (F), HMVEC-lung were stimulated with Mock or Slit2N and subjected to immunofluorescence for VE-cadherin (green). White arrows indicate areas of enhanced VE-cadherin cell surface localization. For all experiments N≧3, * P<0.05, *** P<0.005, **** P<0.001, errors bars represent s.e.m.

FIG. 2. Slit2N enhances a VE-cadherin/p120-catenin interaction. (A), HMVEC-lung were stimulated with IL-1β in the presence of Mock or Slit2N and immunostained for VE-cadherin and p120-catenin. White arrows indicate cell surface areas lacking VE-cadherin or p120-catenin in Mock treated cells. Yellow arrows indicate areas of enhanced cell surface localization of VE-cadherin or p120-catenin in Slit2N treated cells. (B), HMVEC-lung were stimulated with IL-1β in the presence of Mock or Slit2N. Lysates were subjected to immunoprecipitation for VE-cadherin followed by immunoblot for p120-catenin and VE-cadherin. (C), HMVEC-lung were stimulated with IL-1β in the presence of Mock or Slit2N. VE-cadherin internalization (green) was assessed and areas of internalization are indicated by white arrows. (D), in vitro permeability was measured in the presence of a control IgG or VE-cadherin antibody. For all experiments N≧3, * P<0.05, ** P<0.01, *** P<0.005, errors bars represent s.e.m.

FIG. 3. Slit2N inhibits LPS-induced permeability, protein exudates, and cell infiltrates in vivo. (A), Robo4^{+/+} and Robo4^{AP/AP} mice were given an intravenous injection of Mock or Slit2N followed by intratracheal instillation of 10 μg LPS. Mice later received an intravenous injection of Evans Blue Albumin (EBA) and EBA accumulation in the lungs was used to assess vascular permeability (N≧4). (B-D), Alternatively, twenty four hours after LPS administration, bronchoalveolar lavages were obtained and assessed for protein content (B), total inflammatory cell accumulation (C), or neutrophil accumulation (D) (N≧5). e, H&E staining was performed on lung sections from mice exposed to LPS in the presence of Mock or Slit2N. (F), protein exudates measured in mice treated with 3.3 μg LPS(N=5) g-i, mice were given an intravenous injection of Mock or Slit2N with control or VE-cadherin blocking antibody followed by intratracheal instillation of LPS. Bronchoalveolar lavages were obtained and assessed for protein content (G), total inflammatory cell accumulation (H), or neutrophil accumulation (N≧5). * P<0.05, *** P<0.005, **** P<0.001, error bars represent s.e.m.

FIG. 4. Slit2N reduces permeability and mortality in a cecal ligation and puncture model of sepsis. (A), Mice were subjected to CLP or sham operation. Mice were given an intravenous injection of Evans Blue Albumin (EBA) and EBA accumulation measured in the kidney (A) or spleen (B) to assess vascular permeability (N=5). (C), Robo4^{+/+} mice were subjected to CLP and treated with Mock or Slit2N and survival assessed (Mock treated N=15, Slit2N treated N=14). Cytokine (D) or chemokine (E) levels in the serum of Mock or Slit2N treated CLP mice (N=6). (F), Robo4^{AP/AP} mice were subjected to CLP and treated with Mock or Slit2N and survival assessed (Mock treated N=13, Slit2N treated N=13). * P<0.05, ** P<0.01, **** P<0.001, error bars represent s.e.m.

FIG. 5. Slit2N reduces mortality in models of H5N1 infection. (A), Balb/c mice were infected intranasally with H5N1 virus. Mice were given an intravenous injection of Evans Blue Albumin (EBA) and EBA accumulation was measured in the lungs to assess vascular permeability (N=5). (B), mouse survival after H5N1 infection (Mock treated N=20, Slit2N treated N=20). (C), H&E staining was performed on lung sections from H5N1 infected mice 6 days after infection. White arrows in the upper left panel indicate accumulation of edema fluid around a pulmonary arteriole. The upper middle panel demonstrates exuberant alveolar inflammation. The black arrow in the upper right panel indicates the presence of foamy macrophages. (D), H5N1 viral titers were measured 6 days post-infection (N=3 groups of pooled mice). Cytokine (E) or chemokine (F) levels measured in lung homogenates 6 days post-infection (N=3 groups of pooled mice).

FIG. 6. Slit reduces vascular leak caused by multiple inflammatory stimuli through enhancing VE-cadherin at the cell surface. (A), Under normal conditions, alveolar capillaries provide a semi-permeable barrier. (B), Inflammatory stimuli cause a large release of cytokines leading to internalization of VE-cadherin and disruption of barrier function. This results in vascular leak and accumulation of protein-rich edema fluid in the alveolar space. (C), Slit enhances vascular barrier function against multiple cytokines by enhancing VE-cadherin at the cell surface.

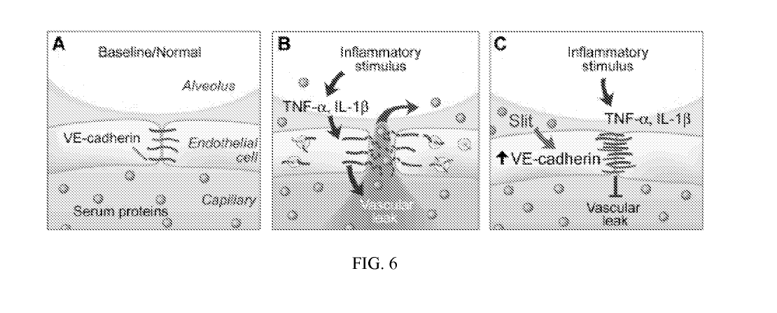

FIG. 7. Recombinant Slit peptides as small as Slit2-D1 (40 kD) are active. In FIG. 7A, different constructs for the Slit protein are depicted. The four leucine rich domains (LRR), the epidermal growth factor homology region (EGF) and the c-terminal tags (MYC/HIS) are indicated. Inhibition of VEGF mediated endothelial cell migration by the different Slit constructs (2 nM) is shown in FIG. 7B.

FIG. 8. SecinH3 inhibits Bleomycin-induced fibrosis. 6-8 week old BL/6 mice were given an intranasal instillation of saline or 0.05 U Bleomycin. 100 uL of Vehicle or 30 uM SecinH3 was administered twice a day via intraperitoneal injection. Pulmonary fibrosis was assessed by Sircol collagen assay. N≧7 animals per group, * P<0.05.

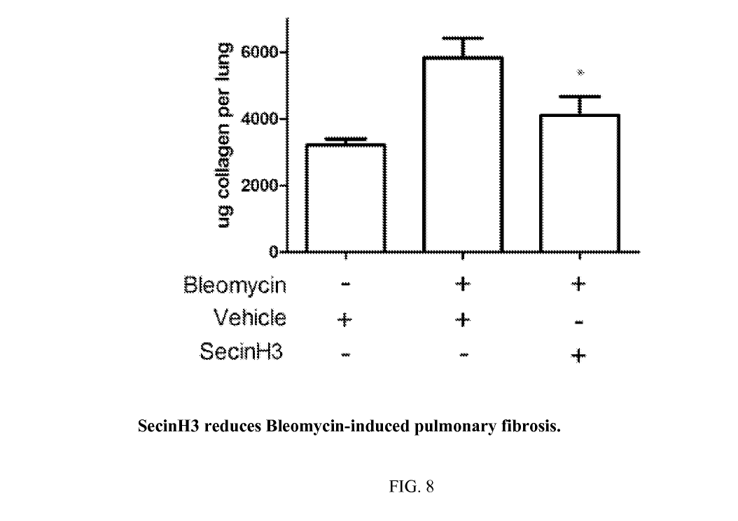

FIG. 9. The chemical structure of SecinH3.

FIG. 10. An illustration of the Robo4 signaling pathway.

FIG. 11. Robo4 expression is increased in the lung 6 hours after LPS instillation.

FIG. 12. The effect of administering a Slit2 protein on the survival of mice infected with Avian Flu Virus in accordance with a mouse model of avian flu.

FIG. 13. (a) Slit2 significantly reduced Bleomycin-induced EBA accumulation in the lung of Robo4^{+/+} mice, eleven days after Bleomycin administration. The effect of Slit2 was lost in Robo4^{AP/AP} mice. (b) Slit2 also significantly reduced Bleomycin-induced pulmonary fibrosis. This effect was lost in Robo4^{AP/AP} mice, indicating that Slit2 acted directly upon the endothelium to reduce pulmonary fibrosis. (c) Histologic examination of the lung using a trichrome stain to enhance the visualization of collagen deposition confirmed the effect of Slit2 in a Robo4-dependent manner.

FIG. 14. Slit inhibits LPS-induced cell infiltrates in a dose-dependent manner. (A) Robo4 or control siRNA knockdown HMVEC-lung were assessed for Robo4 expression by immunoblot. (B) Confocal images of the Z-axis are shown below and to the side as indicated by the yellow lines. Enhanced junctional thickness is observed in Slit2N treated cells. (C-D) Mice were subjected to LPS-induced ALI in the presence of increasing levels of Slit2N. Cell infiltrate and neutrophil are shown. N=4, *P<0.05.

FIG. 15. Slit2N reduces LPS-induced ALI as assessed by quantitative histology. Robo4^{+/+} and Robo4^{AP/AP} mice were subjected to LPS-induced ALI in the presence of Mock of Slit2N. Lung sections from these mice were stained for H&E and quantitative histology to assess the degree of lung injury performed by blinded investigators. N=3, *** P<0.005.

FIG. 16. Slit does not reduce migration of primary human PMNs. (A) Cells were subjected to migration to the leukocyte chemoattractant fMLP in the presence of Mock Slit2. (B) RNA was isolated from hPMNs and subjected to quantitative PCR. Brain cDNA was used as a positive control. N=3, ***P<0.005, error bars represent s.e.m.

FIG. 17. Slit2 protein is expressed throughout the lung in close proximity to the endothelium. Slit2 co-localizes with Robo4 alkaline phosphatase (AP) expression indicated by arrows.

FIG. 18. Significant lung injury is absent during CLP. (A-C) Mice were subjected to CLP and treated with Mock or Slit2N. Lung sections were stained with H&E (A) and quantitative histology to assess the degree of lung injury performed (B) N=3. (C) Permeability in the lungs of mice was assessed using Evans Blue Albumin. N=5.

FIG. 19. Loss of Robo4 does not affect patterning of the vascular endothelium in the early developing lung. A-C, D-F and G-I show Robo4^{+/+}, Robo4^{AP/AP} and Robo4^{AP/AP}˜E12.5 lungs, respectively, stained for epithelium (E-cadherin) and vasculature (CD31). Arrows indicate the distal extent of the left pulmonary artery EC tube. B, E, and H are magnified views of the distal branches of the first left lateral secondary airway branch with the crossbar denoting the thickness of the CD31+ plexus compartment extending linearly outward from the vertex of the distal branches. C, F, and I show magnified view of the distal left pulmonary artery EC tube regions stained with an antibody directed against CD31. The locations of the first, second and third left dorsal secondary airway buds are denoted by D1, D2 and D3.

FIG. 20. Loss of Robo4 does not affect patterning of the vascular endothelium in the developing lung. A-B, C-D and E-F show Robo4^{+/+}, Robo4^{+/AP} and Robo4^{AP/AP}˜E14.5 lungs, respectively, stained for lung epithelium (E-cadherin) and vascular development (CD31). Arrows denote the left pulmonary artery located lateral to the left primary bronchus. The dark arrowheads in A, C, and E mark the branch of the right pulmonary artery supplying the right accessory lobe and located posterior to the right secondary airway branch to the accessory lobe.

### DETAILED DESCRIPTION

### I. Definitions

Disclosed are materials, compositions, and components that can be used for, can be used in conjunction with, can be used in preparation for, or are products of the disclosed active agents, compositions and methods. These and other materials are disclosed herein, and it is understood that when combinations, subsets, interactions, groups, etc. of these materials are disclosed that, while specific reference of each various individual and collective combinations and permutation of these compounds may not be explicitly disclosed, each is specifically contemplated and described herein. For example, if a polypeptide is disclosed and discussed and a number of modifications that can be made to a number of molecules including the polypeptide are discussed, each and every combination and permutation of polypeptide and the modifications that are possible are specifically contemplated, unless specifically indicated to the contrary. Thus, if a class of molecules A, B, and C are disclosed as well as a class of molecules D, E, and F, and an example of a combination molecule, A-D, is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, equivalents to the specific embodiments of the method and compositions described herein. Such equivalents are intended to be encompassed by the included claims.

It is also to be understood that the terminology used herein is for the purpose of describing particular embodiments only, and is not intended to limit the scope of the inventions described herein.

Unless defined otherwise, all technical and scientific terms used herein have the meanings that would be commonly understood by one of skill in the art in the context of the present specification.

It must be noted that as used herein and in the appended claims, the singular forms a, an, and the include plural reference unless the context clearly dictates otherwise. Thus, for example, reference to a polypeptide includes a plurality of such polypeptides, reference to the polypeptide is a reference to one or more polypeptides and equivalents thereof known to those skilled in the art, and so forth.

Optional or optionally means that the subsequently described event, circumstance, or material may or may not occur or be present, and that the description includes instances where the event, circumstance, or material occurs or is present and instances where it does not occur or is not present.

Ranges can be expressed herein as from about one particular value, and/or to about another particular value. When such a range is expressed, another embodiment includes from the one particular value and/or to the other particular value. Similarly, when values are expressed as approximations, by use of the antecedent about, it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as about that particular value in addition to the value itself. For example, if the value 10 is disclosed, then about 10 is also disclosed. It is also understood that each unit between two particular units are also disclosed. For example, if 10 and 15 are disclosed, then 11, 12, 13, and 14 are also disclosed.

Alkyl refers to an optionally substituted hydrocarbon group joined by single carbon-carbon bonds and having 1 to 8 carbon atoms joined together. The alkyl hydrocarbon group may be straight-chain or contain one or more branches. These groups include methyl, ethyl, propyl, isopropyl, butyl, isobutyl, tertiary butyl, pentyl, hexyl, and the like. Lower alkyl refers to optionally substituted branched- or straight-chain alkyl having 1 to 4 carbons.

Alkenyl refers to an optionally substituted hydrocarbon group containing at least one carbon-carbon double bond between the carbon atoms and containing 2-8 carbon atoms joined together. The alkenyl hydrocarbon group may be branched or straight-chain.

Cycloalkyl refers to an optionally substituted cyclic alkyl or an optionally substituted non-aromatic cyclic alkenyl and includes monocyclic and multiple fused ring structures such as bicyclic and tricyclic. The cycloalkyl may have, for example, 3 to 15 carbon atoms. In one embodiment, cycloalkyl has 5 to 12 carbon atoms. Examples of suitable cycloalkyl groups include cyclopropyl, cyclobutyl, cyclopentyl, cyclohexyl and the like.

Heterocycle refers to optionally substituted saturated or partially saturated non-aromatic ringed moieties including at least one non-carbon atom. Heterocyclic moieties typically comprise a single ring or multiple fused ring structures, such as bicyclic and tricyclic. In one embodiment, the ring(s) is 5 to 6-membered and typically contains 1 to 3 non-carbon atoms. Non-carbon atoms for heterocycle may be independently selected from nitrogen, oxygen and sulfur.

Aryl refers to an optionally substituted aromatic group with at least one ring having a conjugated pi-electron ring system, and includes monocyclic and multiple fused ring structures such as bicyclic and tricyclic. Aryl includes optionally substituted carbocyclic aryl. Examples of suitable aryl groups include phenyl, naphthyl, anthracenyl, phenanthrenyl and the like.

Heterocyclic aryl refers to an optionally substituted aromatic group with at least one ring having a conjugated pi-electron ring system including at least one non-carbon atom. Heterocyclic aryl moieties typically comprise one ring or multiple fused ring structures, such as bicyclic and tricyclic. Examples of suitable heterocyclic aryl groups include furanyl, thienyl, pyrrolyl, imidazolyl, pyridinyl, and the like.

Alkoxy refers to oxygen joined to an alkyl group. Lower alkoxy refers to oxygen joined to a lower alkyl group. In one embodiment, the oxygen is joined to an unsubstituted alkyl 1 to 4 carbons in length. For example, the alkoxy may be methoxy, ethoxy and the like.

Alkylene refers to an optionally substituted hydrocarbon chain containing only carbon-carbon single bonds between the carbon atoms. The alkylene chain has 1 to 6 carbons and is attached at two locations to other functional groups or structural moieties. Examples of suitable alkylene groups include methylene, ethylene and the like.

As used herein, small molecule refers to low molecular weight compounds. For example, in particular embodiments, such small molecule compounds are compounds the exhibit a molecular weight of between 50 daltons to 800 daltons. In alternative embodiments, a small molecule as described herein exhibit a molecular weight selected from the ranges of between 100 daltons and 500 daltons and between 250 daltons to 475 daltons.

As used herein, the term subject means any target of administration. The subject can be a vertebrate, for example, a mammal. Thus, the subject can be a human. The term does not denote a particular age or sex. Thus, adult and newborn subjects, as well as fetuses, whether male or female, are intended to be covered. The term patient refers to a subject afflicted with a pathologic condition. The term patient includes human and veterinary subjects.

Inhibit, inhibiting, and inhibition mean to prevent, decrease, inactivate, or reverse an activity, response, condition, disease, or other biological parameter. Inhibit, inhibiting, and inhibition can include, but is not limited to the complete ablation of the activity, response, condition, or disease. Inhibit, inhibiting, and inhibition can also include, for example, a slowing or reduction of an activity, response, condition, disease, or other biological parameter as compared to a native level, with the term native level referring to a level evident in the absence of an inhibiting agent. Inhibit, inhibiting, and inhibition can also include, for example, reversal of an activity, response, condition, disease, or other biological parameter as compared to a native level, with the term native level referring to a level evident in the absence of an inhibiting agent. In this context, a reduction can be any measurable reduction. In particular embodiments, a reduction can be, for example, a 10%, 20%, 30%, 40%, 50%, 60%, 70%, 80%, 90%, 100%, or any amount of reduction in between the specifically recited percentages, as compared to a native level.

Promote, promotion, and promoting refer to a preservation, restoration, or increase in an activity, response, condition, or other biological parameter. Promote, promotion, and promoting can include but is not limited to the initiation of an activity, response, condition, or biological parameter. Alternatively, promote, promotion, and promoting can include preservation of an activity, response, condition, or other biological parameter in light of a condition that would otherwise degrade, reduce or eliminate the relevant activity, response, condition, or other biological parameter. Promote, promotion, and promoting can also include, for example, an increase in the activity, response, condition, or biological parameter as compared to a native or control level. In particular embodiments, the increase in an activity, response, condition, or other biological parameter can be an increase of 10%, 20%, 30%, 40%, 50%, 60%, 70%, 80%, 90%, 100%, or more, including any amount of increase in between the specifically recited percentages, as compared to native or control levels, with the term native level referring to a level evident in the absence of an promoting agent.

The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be used in providing a pharmaceutical formulation and can be selected to minimize any degradation of the active agent and to minimize any adverse side effects in the subject.

As used herein, the terms treat, treating, and treatment refer to a therapeutic benefit, whereby the detrimental effect(s) or progress of a particular pathologic condition, disease, condition, event or injury is prevented, reduced, halted, reversed or slowed.

A therapeutically effective amount is the amount of compound which achieves a therapeutic benefit, such as, for example, by inhibiting or reversing an activity, response, condition, disease, or other parameter associated with a pathologic condition. A therapeutically effective amount may be an amount which relieves, at least to some extent, one or more symptoms of a pathologic condition in a subject; returns to normal, either partially or completely, one or more physiological or biochemical parameters associated with or causative of a pathologic condition; and/or reduces the likelihood of the onset of a pathologic condition.

The terms pathologic or pathologic conditions refer to any deviation from a healthy, normal, or efficient condition which may be the result of a disease, condition, event or injury.

As the terms are used herein, protein and peptide refer to polypeptide molecules generally and are not used to refer to polypeptide molecules of any specific size, length or molecular weight. Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are well understood in the art and are those in which at least one residue has been removed and a different residue inserted in its place. Typically, substitutions made in the formation of substitutional variants are conservative substitutions, as are well known in the art, and often substitutional variants may be made to made to enhance one or more characteristics of a polypeptide molecule, such as, for example, circulating half-life, stability, etc., while retaining or improving the biologic activity of polypeptide.

Substantial changes in function or immunological identity are made by selecting substitutions differ in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

In the context of recombinantly produced polypeptides, certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983], incorporated herein by reference), acetylation of the N-terminal amine and, in some instances, amidation of the C-terminal carboxyl.

It is understood that one way to define the derivatives, analogs and homologs of the polypeptides disclosed herein is through defining the derivatives, analogs and homologs in terms of homology/identity to specific known sequences. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

It is understood that there are numerous amino acid and peptide analogs which can be incorporated into the disclosed polypeptides. For example, there are numerous D amino acids or amino acids which have a different functional substituent then the amino acids shown in Table 1. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference).

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

As used herein, vascular permeability refers to the capacity of small molecules (e.g., ions, water, nutrients), large molecules (e.g., proteins and nucleic acids) or even whole cells (lymphocytes on their way to the site of inflammation) to pass through a blood vessel wall.

### II. Robo4 Signaling Pathway

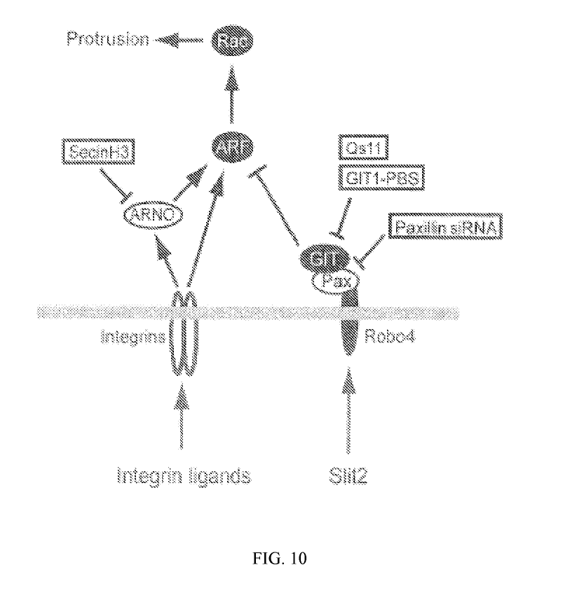

A signaling pathway whereby Robo4 signaling inhibits pathologic angiogenesis and neovascularization is described, in International Publication No. WO 2009/129408, International Publication No. WO 2008/073441, and Jones et al. (C. A. Jones et al., 2008. Robo4 stabilizes the vascular network by inhibiting pathologic angiogenesis and endothelial hyperpermeability. Nat Med 14:448-453). As is described in these references, expression of Robo4 confers responsiveness to Slit2, and Slit2-Robo4 signaling negatively regulates cellular protrusive activity stimulated by cell adhesion. Such negative regulation is mediated by interaction of Robo4 with the adaptor protein, paxillin, and its paralogues, which recruits ARF-GAPs such as GIT1, leading to local inactivation of ADP ribosylation factor 6 (ARF6). This signaling pathway (illustrated in FIG. 10) thereby interferes with adhesion-mediated Rac1 activation and cell protrusion. The signaling pathway described in FIG. 10 presents multiple targets for modulating the Robo-4 signaling pathway, and Jones et al., International Publication No. WO 2009/129408, and International Publication No. WO 2008/073441, further describe that modulation of ARF-GAPs and ARF-GEFs involved in the Robo4 signaling pathway can be accomplished without Slit/Robo4 signaling. The contents of each of Jones et al., International Publication No. WO 2009/129408, and International Publication No. WO 2008/073441 are incorporated herein by this reference.

Robo4 signaling works to preserve vascular integrity in the presence of multiple different mediators of inflammation. For example, the Robo-4 signaling pathway can be utilized to preserve vascular integrity in the presence of endotoxin (e.g., lipopolysaccharide or “LPS”), tumor necrosis factor (e.g., TNF-α), and interleukin-1β (“IL-1β”), each of which is a known mediators of inflammation (Dinarello, C. A. 1997. Proinflammatory and anti-inflammatory cytokines as mediators in the pathogenesis of septic shock. Chest 112:321 S-329S). Moreover, the Robo-4 signaling pathway functions to preserve endothelial barrier function in multiple different tissues, such as, for example, in the lung, the kidney, and the spleen. Even further, the Robo4 signaling pathway serves not only to preserve vascular integrity in conditions associated with an acute inflammatory response, but also the Robo-4 signaling pathway can be utilized to preserve endothelial barrier function in-vivo in models of acute and chronic pulmonary inflammation.

### II. Active Agents & Compositions

The active agents and compositions described herein serve to promote vascular barrier function. In each embodiment, the compositions described herein include at least one active agent capable of promoting vascular barrier function, and in one such embodiment, the compositions described herein include an active agent that promotes the barrier function of vascular endothelium. In another embodiment, the compositions described herein include an active agent that promotes vascular barrier function in endothelial tissue selected from one of vascular endothelium of the lung, vascular endothelium of the kidney and vascular endothelium of the spleen. In another embodiment, a composition as described herein includes an active agent that inhibits vascular permeability associated with conditions leading to acute pulmonary inflammation as well as conditions associated with chronic pulmonary inflammation, such as the development and progression of pulmonary fibrosis. As illustrated by the experimental examples provided herein, active agents according to the present description, in particular embodiments, promote vascular barrier function even in the presence of multiple mediators of inflammation and vascular permeability, including, for example, endotoxins (e.g., LPS), tumor necrosis factor (e.g., TNF-α), and IL-1β.

In specific embodiments, the active agents and compositions described herein are suitable for treating a subject suffering from a pathological condition such as pulmonary fibrosis, as well as other conditions associated with acute pulmonary vascular inflammation. In one such embodiment, a composition as described herein includes an active agent that inhibits one or more of acute pulmonary vascular edema, acute pulmonary vascular inflammation, and chronic pulmonary vascular inflammation associated with development or progression of pulmonary fibrosis, including idiopathic pulmonary fibrosis. In another such embodiment, a composition as described herein includes an active agent that promotes vascular barrier function in animals, including humans, exposed to a microbial endotoxin, or suffering from an influenza infection, such as an avian flu infection. In still further embodiments, compositions described herein include an active agent that promotes vascular barrier function and inhibits vascular permeability associated with pathological conditions such as bacterial sepsis, or influenza infection, such as an avian flu infection.

In the vascular endothelium, critical stabilizing interactions are mediated by the adherens junction protein, vascular endothelial cadherin (VE-cadherin) (Dejana, E., F. Orsenigo, and M. G. Lampugnani. 2008. The role of adherens junctions and VE-cadherin in the control of vascular permeability. J Cell Sci 121:2115-2122; Vestweber, D. 2008. VE-cadherin: the major endothelial adhesion molecule controlling cellular junctions and blood vessel formation. Arterioscler Thromb Vasc Biol 28:223-232). VE-cadherin surface expression is regulated by the association of p120-catenin with VE-cadherin, and the association of p120-catenin with VE-cadherin is known to inhibit VE-cadherin internalization from the cell surface and promote vascular stability (Potter, M. D., S. Barbero, and D. A. Cheresh. 2005. Tyrosine phosphorylation of VE-cadherin prevents binding of p120- and beta-catenin and maintains the cellular mesenchymal state. J Biol Chem 280:31906-31912; Xiao, K., J. Garner, K. M. Buckley, P. A. Vincent, C. M. Chiasson, E. Dejana, V. Faundez, and A. P. Kowalczyk. 2005. p120-Catenin regulates clathrin-dependent endocytosis of VE-cadherin. Mol Biol Cell 16:5141-5151). In particular embodiments, the active agents described herein promote the presence of VE-cadherin at cell surface junctions. In one such embodiment, an active agent according to the present description promotes cell surface p120-catenin expression. In promoting expression of cell surface p120-catenin, it is presently believed, without being bound by a particular theory, that such embodiments preserve the association between VE-cadherin and p120-catenin and, thereby, promote vascular integrity by reducing VE-cadherin endocytosis, such as may otherwise occur in the presence of one more mediators of inflammation (e.g., one or more cytokines). Therefore, in particular embodiments, the active agents described herein promote vascular barrier function in animals, including humans, by one or both of promoting the presence of VE-cadherin at the surface of endothelial cells, such as vascular endothelial cells, and promoting expression of p120-catenin at the surface of endothelial cells, such as vascular endothelial cells

An active agent according to the present description can include a Slit polypeptide, such as Slit2 polypeptide. When discussing Slit2 polypeptides as contemplated herein, full-length Slit2 proteins, as well as derivatives, analogs and homologs of full-length Slit2 proteins are contemplated, provided that such polypeptides promote endothelial barrier function in the presence of one or more mediators of inflammation, including one or more of an endotoxin (e.g., LPS), tumor necrosis factor (e.g., TNF-α), and IL-1β, inhibit vascular leak in the presence of one or more mediators of inflammation, including one or more of an endotoxin (e.g., LPS), tumor necrosis factor (e.g., TNF-α), and IL-1β, promote of the presence of VE-cadherin at the surface of vascular endothelial cells, or promote expression of p120-catenin at the surface of vascular endothelial cells. A derivative polypeptide molecule refers to a polypeptide formed from native compounds either directly or by modification or partial substitution. A homolog polypeptide molecule refers to a polypeptide product of a particular gene derived from a different species. An analog polypeptide molecule is a polypeptide that is similar in structure, but not identical, and differs with respect to number or nature of amino acids included in a referenced polypeptide sequence. For example, an analog to a given polypeptide will exhibit a level of sequence homology, but may include one or more amino acid substitutions or deletions.

In specific embodiments, where the active agent is a Slit2 polypeptide, the active agent may be selected from mammalian Slit2 polypeptides, such as a human Slit2 polypeptide. Where the active agent is a mammalian Slit2, the active agent may be selected from known, full-length, naturally occurring, mammalian Slit2 polypeptides, such as, for example, the Slit2 polypeptide represented by SEQ ID NO: 1, as well as derivative, analogs and homologs thereof, that are capable of on one or more of the following: promoting endothelial barrier function in the presence of one or more mediators of inflammation, including one or more of an endotoxin (e.g., LPS), tumor necrosis factor (e.g., TNF-α), and IL-1β; inhibiting vascular leak in the presence of one or more mediators of inflammation, including one or more of an endotoxin (e.g., LPS), tumor necrosis factor (e.g., TNF-α), and IL-1β; promoting the presence of VE-cadherin at the surface of vascular endothelial cells; and promoting expression of p120-catenin at the surface of vascular endothelial cells. As will be readily appreciated, naturally occurring Slit2 polypeptides can be isolated and purified according to techniques known in the art (Wang, K H et al. 1999. Biochemical purification of a mammalian slit protein as a positive regulator of sensory axon elongation and branching. Cell March 19; 96(6):771-84; Chedotal, A. 2007. Slits and their receptors. Adv Exp Med Biol 621:65-80).

In addition to naturally occurring Slit2 polypeptides, a Slit2 active agent as contemplated herein may be obtained through recombinant or synthetic production techniques well known in the art. Even further, the active agent may be selected from derivatives, analogs, or homologs of naturally occurring, recombinant, or synthetic mammalian Slit2 polypeptides. In specific embodiments, a Slit2 active agent can be selected from a fragment of a naturally occurring Slit2 protein, such as the fragment represented either of SEQ ID NO: 2 or SEQ ID NO: 3, as well as derivatives, analogs and homologs thereof, capable of one or more of the following: promoting endothelial barrier function in the presence of one or more mediators of inflammation, including one or more of an endotoxin (e.g., LPS), tumor necrosis factor (e.g., TNF-α), and IL-1β; inhibiting vascular leak in the presence of one or more mediators of inflammation, including one or more of an endotoxin (e.g., LPS), tumor necrosis factor (e.g., TNF-α), and IL-1β; promoting the presence of VE-cadherin at the surface of vascular endothelial cells; and promoting expression of p120-catenin at the surface of vascular endothelial cells.

In still other embodiments, the Slit2 polypeptides represented by SEQ ID NO: 4 through SEQ ID NO: 12 may be used as an active agent. In particular, an active agent according to the present description may be selected from Slit2N (SEQ ID NO: 4), the Slit2 polypeptide represented by SEQ ID NO: 5, Slit2ΔP (SEQ ID NO: 6), Slit2 D1 (SEQ ID NO: 7), Slit2 D1-D2 (SEQ ID NO: 8), Slit2 D1-D3 (SEQ ID NO: 9), Slit2 D1-D4 (SEQ ID NO: 10), Slit2 D1-E5 (SEQ ID NO: 11), and Slit2 D1-E6 (SEQ ID NO: 12), as well as derivative, analogs and homologs thereof capable of one or more of the following: promoting endothelial barrier function in the presence of one or more mediators of inflammation, including one or more of an endotoxin (e.g., LPS), tumor necrosis factor (e.g., TNF-α), and IL-1β; inhibiting vascular leak in the presence of one or more mediators of inflammation, including one or more of an endotoxin (e.g., LPS), tumor necrosis factor (e.g., TNF-α), and IL-1β; promoting the presence of VE-cadherin at the surface of vascular endothelial cells; and promoting expression of p120-catenin at the surface of vascular endothelial cells.

In still other embodiments, where the active agent is selected from a derivative, analog or homolog of one of the Slit2 polypeptides described herein, the active agent may be selected from a derivative, analog or homolog of a naturally occurring mammalian Slit2 polypeptide, or one of the Slit2 polypeptides described by SEQ. ID. NO: 1, SEQ ID NO: 2, and SEQ ID NO: 4 through SEQ ID NO: 12, with such active agent exhibiting a polypeptide sequence homology of at least about 80%, 81%, 82%, 83%, 84%, 85%, 86%, 87%, 88%, 89%, 90%. 91%, 92%, 93%, 94%, 95%, 96%, 97%, 98%, 99%, or greater, to the relevant naturally occurring mammalian Slit2 polypeptide, or to any one of SEQ. ID. NO: 1, SEQ ID NO: 2, and SEQ ID NO: 4 through SEQ ID NO: 12. In each such embodiment, the derivative, analog or homolog is selected for its capacity for one or more of the following: promoting endothelial barrier function in the presence of one or more mediators of inflammation, including one or more of an endotoxin (e.g., LPS), tumor necrosis factor (e.g., TNF-α), and IL-1β; inhibiting vascular leak in the presence of one or more mediators of inflammation, including one or more of an endotoxin (e.g., LPS), tumor necrosis factor (e.g., TNF-α), and IL-1β; promoting of the presence of VE-cadherin at the surface of vascular endothelial cells; and promoting expression of p120-catenin at the surface of vascular endothelial cells

In another embodiment, the active agent is a derivative, homolog, or analog of a naturally occurring mammalian Slit2 polypeptide, or one of the Slit2 polypeptides described by SEQ. ID. NO: 1, SEQ ID NO: 2, and SEQ ID NO: 4 through SEQ ID NO: 12, yet exhibits less polypeptide sequence homology to the polypeptide from which it is derived, such as, for example, a homology selected from one of 80% or less, 70% or less, 60% or less, or 50% or less, while retaining the capacity for one or more of the following: promoting endothelial barrier function in the presence of one or more mediators of inflammation, including one or more of an endotoxin (e.g., LPS), tumor necrosis factor (e.g., TNF-α), and IL-1β; inhibiting vascular leak in the presence of one or more mediators of inflammation, including one or more of an endotoxin (e.g., LPS), tumor necrosis factor (e.g., TNF-α), and IL-1β; promoting the presence of VE-cadherin at the surface of vascular endothelial cells; and promoting expression of p120-catenin at the surface of vascular endothelial cells.

In one embodiment, an active agent as described herein is a ligand of a Robo4 receptor. In such an embodiment, the ligand of Robo4 can be any molecule that acts through Robo4 to promote vascular barrier function. As used herein, the expression acts through refers to a ligand that has an effect on endothelial cells which requires the presence of the Robo4 receptor. In one embodiment, a ligand effecting endothelial cells may act through Robo4 by binding or associating with a Robo4 receptor in a manner that results in Robo4 signaling. Without being bound by a particular theory, it is presently believed that the Slit2 polypeptides described herein act through the Robo4 receptor. Therefore, in specific embodiments where an active agent according to the present description is a ligand of the Robo4 receptor, the active agent may be selected from a Slit polypeptide described herein. In another such embodiment, the ligand of Robo4 can be any molecule that acts through Robo4 to promote the presence of VE-cadherin at cell surface junctions. In specific embodiments, the Slit ligand, or fragment or variant thereof, binds to or associates with Robo4 in a manner that results in one or more of the following: promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cells.

In yet another embodiment, an active agent as described herein may be a ligand of Robo4, wherein the ligand acts through Robo4 to promote cell surface expression of p120-catenin. In still a further embodiment, an active agent as described herein includes a ligand of a Robo4 receptor, wherein the ligand acts through Robo4 to initiate paxillin activation of GIT1. In another embodiment, an active agent as described herein includes a ligand of a Robo4 receptor, wherein the ligand acts through Robo4 to activate GIT1 inhibition of ARF6. In a further embodiment, an active agent as described herein includes a ligand of a Robo4 receptor, wherein the ligand acts through Robo4 in a manner that results in one or more of the following: promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cells. Where the active agent the present invention includes a ligand of Robo4, in specific embodiments, the ligand can be a molecule that binds the extracellular domain of Robo4 leading to Robo4 signaling.

A polypeptide of a desired structure can be produced using methods and materials well known in the art. For example, various methods for isolating naturally occurring polypeptides or producing recombinant polypeptides are well known. Moreover, various methods are known for synthetically producing a polypeptide of desired sequence. For example, peptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). Slit polypeptides described herein can be obtained through recombinant and synthetic techniques well-known to those of skill in the art, including those described herein and, for example, the methods described in International Publication No. WO 2009/129408, International Publication No. WO 2008/073441.

One skilled in the art can readily appreciate that a peptide corresponding to a desired protein can be synthesized by standard chemical reactions. For example, a peptide can be synthesized and not cleaved from its synthesis resin whereas another peptide fragment of a protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, a desired protein or peptide can be synthesized in-vivo using standard recombinant techniques. Where independent peptides that are to be linked to form a desired protein are independently produced in-vivo, once such independent peptides are produced and isolated, they may be linked to form a desired protein or fragment thereof via similar peptide condensation reactions.

For example, enzymatic ligation of cloned or synthetic peptide segments allow relatively short peptide fragments to be joined to produce larger peptide fragments, polypeptides or whole protein domains. (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction. (Dawson et al. Synthesis of Proteins by Native Chemical Ligation. Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

In another embodiment, an active agent as described herein may be a small molecule active agent that inhibits the activity of a cytohesin selected from the ARNO family of cytohesins. Small molecule active agents selected from compounds that inhibit the availability, activation or activity of an ARF-GEF, such as a cytohesin, a cytohesin selected from the ARNO family of cytohesins, or ARNO, in a manner that results in inhibition of one or more ARFs, such as ARF6 and ARF1 are described in Jones et al., International Publication No. WO 2009/129408, and International Publication No. WO 2008/073441. In the context of the compositions and methods described herein, it has been determined that small molecule active agents that inhibit the availability, activation or activity of an ARF-GEF, such as a cytohesin, a cytohesin selected from the ARNO family of cytohesins, or ARNO, in a manner that results in inhibition of one or more ARFs, such as ARF6 and ARF1, can inhibit pulmonary vascular permeability and/or inflammation and the pulmonary fibrosis that can develop as a result.

In specific embodiments, a small molecule active agent as described herein inhibits the activity of a cytohesin selected from the ARNO family of cytohesins in a manner that results in one or more of the following: inhibition of the activity or availability of ARF6; inhibition of the activity or availability of ARF1; promotion of vascular endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cells. In another embodiment, the small molecule active agent inhibits the activity of ARNO in a manner that results in one or more of the following: inhibition of ARF6; preservation of vascular endothelial barrier function; promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cells. In yet another embodiment, the small molecule active agent inhibits the activity or availability of ARF6, resulting in one or more the following: preservation of vascular endothelial barrier function; promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cells.

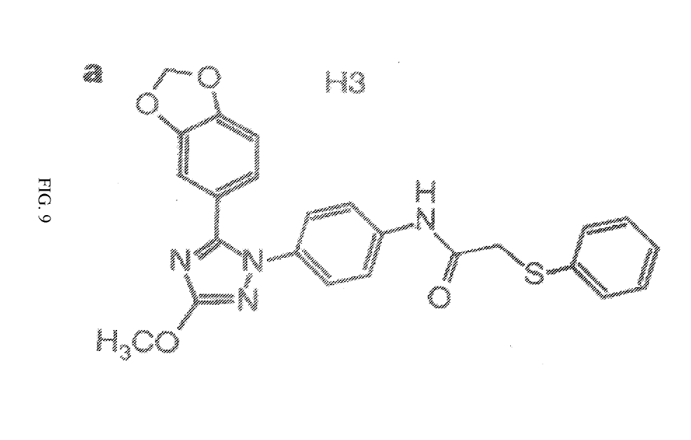

In a specific embodiment, an active agent as described herein may be SecinH3, the structure of which is provided in FIG. 9. SecinH3 is an inhibitor of cytohesins (see, for example, Hafner et al, Inhibition of cytohesins by SecinH3 leads to hepatic insulin resistance, Nature (2006), 444, 941-944, and International Patent App. Publication No. WO 2006/053903, the contents of both of which are incorporated herein by reference). It has been found that Secin-H3 inhibits the effects of mediators of inflammation and vascular permeability. Thus, in one embodiment, SecinH3 may be selected as a small molecule active agent that inhibits the activity of a cytohesin selected from the ARNO family of cytohesins in a manner that results in inhibition of an ARF selected from ARF6 and ARF1, and provides one or more of the following: promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cells.

In another embodiment, a composition as described herein includes one or more small molecule active agents selected from compounds that inhibit the availability, activation or activity of an ARF-GEF, such as a cytohesin, a cytohesin selected from the ARNO family of cytohesins, or ARNO in a manner that results in one or more of the following: inhibition of the activity or availability of ARF6; inhibition of the activity or availability of ARF1; preservation of vascular endothelial barrier function; promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cells.

Where an active agent according to the present description includes a small molecule active agent, in specific embodiments, the active agent may include one or more compounds having the following chemical formula (Formula 1):

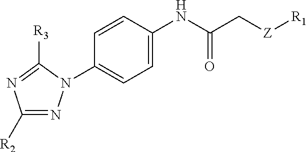

wherein:

R¹ and R³ are independently chosen from optionally substituted aryl, optionally substituted heteroaryl, optionally substituted cycloalkyl, or optionally substituted heterocycle;

R² is chosen from hydrogen, lower alkoxy, lower alkyl, halogen or hydroxy;

Z is chosen from O, S, NH, alkylene or a single bond; or

pharmaceutically acceptable salts, solvates or hydrates thereof.

In one such embodiment, the one or more compounds are selected from compounds as described by Formula 1, wherein R³ is substituted with 1 to 5 substituents independently chosen from halogen, lower alkyl, lower alkoxy, heteroatom lower alkyl, hydroxy, or methylene dioxy, wherein two substituents together may form a fused cycloalkyl or heterocyclic ring structure. In another such embodiment, the one or more compounds are selected from compounds as described by Formula 1, wherein R¹ is chosen from unsubstituted aryl or unsubstituted heteroaryl; R² is chosen from hydrogen, lower alkoxy, or lower alkyl; R³ is chosen from aryl, optionally substituted with 1 to 5 substituents independently chosen from halogen, lower alkyl, lower alkoxy, or methylene dioxy, wherein two substituents together may form a fused cycloalkyl or heterocyclic ring structure; and Z is chosen from O, S, or a single bond.

In another embodiment, where an active agent according to the present description includes a small molecule active agent, the active agent may be selected from one or more compounds having the following chemical formula (Formula 2):

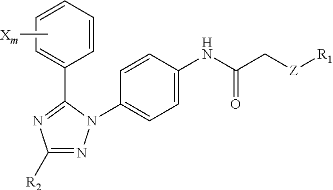

wherein:

R¹ is chosen from optionally substituted aryl, optionally substituted heteroaryl, optionally substituted cycloalkyl, or optionally substituted heterocycle;

R² is chosen from hydrogen, lower alkoxy, lower alkyl, halogen or hydroxy;

Z is chosen from O, S, NH, alkylene or a single bond;

X is independently chosen from halogen, lower alkyl, lower alkoxy, heteroatom lower alkyl, hydroxy, or methylene dioxy, wherein two substituents together may form a fused cycloalkyl or heterocyclic ring structure;

m is 0 to 5; or

pharmaceutically acceptable salts, solvates or hydrates thereof.

In one such embodiment, the one or more compounds are selected from the following compounds:

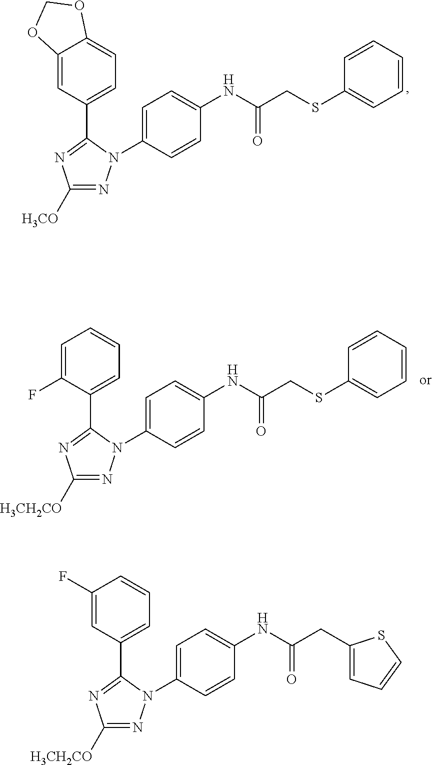

or pharmaceutically acceptable salts, solvates or hydrates thereof.

As described in Jones et al., International Publication No. WO 2009/129408, and International Publication No. WO 2008/073441, it is believed that activation of Robo4 results in interaction between Robo4 with the adaptor protein, paxillin, and its paralogues, which recruits ARF-GAPs, such as GIT1, leading to local inactivation of ARF6. It is further described in Jones et al., International Publication No. WO 2009/129408, and International Publication No. WO 2008/073441, modulation of AFR-GAPs and ARF-GEFs can be accomplished without Robo4 signaling. Without being bound by a particular theory, it is presently believed that the small molecule active agents described herein may function, at least in part, by achieving the benefits of Robo4 signaling, such as those described herein and in Jones et al., International Publication No. WO 2009/129408, and International Publication No. WO 2008/073441, without requiring the use of a Robo4 ligand or direct activation of Robo4.

Compositions including an active agent as described herein are also provided. Such compositions may include one or more active agents as described herein. In one embodiment, a composition is prepared as a pharmaceutical formulation. For example, in addition to one or more active agent as described herein, a pharmaceutical formulation may include a pharmaceutically acceptable carrier and/or one or more pharmaceutically acceptable excipients to provide a formulation that is suitable for therapeutic administration. As used herein, pharmaceutically acceptable refers to a material that is not biologically or otherwise undesirable, e.g., the material is suitable for administration to a subject together with the desired active agent (e.g., a desired active agent as described herein) and is compatible with other components of the pharmaceutical formulation in which it is contained. The carrier and any excipient(s) would naturally be selected to minimize any degradation of the active agent or adverse side effects in the subject.

A pharmaceutical formulation according to the present description may be prepared in any form suitable for administration, such as, by way of example, a tableted composition, a powder composition for encapsulation, a solution composition for direct ingestion, encapsulation or parenteral delivery, an emulsion, a gel, a cream, suppository, or a suspension, such as a formulation that incorporates or is incorporated into, for example, microparticles, a matrix material, or liposomes. A pharmaceutical formulation as described herein may include components targeted to a particular cell type via antibodies, receptors, or receptor ligands. Pharmaceutical carriers and excipients and their formulations are well described in the literature, including, for example, in Remington: The Science and Practice of Pharmacy (19th ed.) ed. A. R. Gennaro, Mack Publishing Company, Easton, Pa. 1995.

Where appropriate, a pharmaceutically-acceptable salt or other tonicity modifying agent may be used in the pharmaceutical formulation to render the formulation isotonic. Examples of liquid pharmaceutically-acceptable carriers include, but are not limited to, saline, Ringer's solution, and dextrose solution. Where the pharmaceutical formulation is provided as a solution or suspension, particularly for parenteral delivery, the pH of the formulation can be adjusted as desired to facilitate delivery to a subject and/or preservation of the active agent or other formulation components. Carriers and excipients suitable for preparing pharmaceutical formulations include, for example, a well-known variety of pharmaceutically acceptable polymers, saccharides, salts, lipids, phospholipids, surfactants, gels, polypeptides, and amino acids. The pharmaceutical formulation according to the present description may include sustained release preparations. It will be apparent to those persons skilled in the art that certain carriers and/or excipients may be more preferable depending upon, for instance, the route of administration and concentration of composition being administered. A pharmaceutical formulation as described herein may include one or more thickener, flavoring, diluent, buffer, preservative, antimicrobial agents, antiinflammatory agents, anesthetics, surface active agent, and the like.

The active agents and compositions may be administered as a pharmaceutically acceptable acid- or base-addition salt. Where that is the case, the desired salt may be formed by reaction with an inorganic acid, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxyide, ammonium hydroxide, potassium hydroxide, and organic bases such as mono-, di-, trialkyl and aryl amines and substituted ethanolamines.

The compositions disclosed herein, including pharmaceutical formulations, may be administered in a number of ways depending on whether local or systemic treatment is desired, and on the area to be treated. Parenteral administration of the composition, if used, is generally characterized by injection. Injectables can be prepared in conventional forms, either as liquid solutions or suspensions, solid forms suitable for dissolution or suspension in liquid prior to injection, or as emulsions. A revised approach for parenteral administration involves use of a slow release or sustained release system such that a constant dosage is maintained. (See, e.g., U.S. Pat. No. 3,610,795, which is incorporated by reference herein.)

The exact amount of a given composition required to achieve a therapeutic affect will vary from subject to subject, depending on the species, age, weight and general condition of the subject, the severity of the pathologic condition being treated, the particular active agent used, its mode of administration, and the like. The dosage ranges for the administration of the compositions are those large enough to produce a therapeutic effect. The dosage can be adjusted to avoid or reduce the occurrence of adverse side effects, such as unwanted cross-reactions, anaphylactic reactions, and the like. The dosage may vary with the age, condition, sex and extent of the disease in the patient, route of administration, or whether other drugs are included in the regimen. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

### III. Methods

Methods for promoting vascular endothelial barrier function are provided herein. In one embodiment, a method for promoting vascular endothelial barrier function includes treating one or more vascular endothelial cells with an active agent as described herein. In one such embodiment, the step of treating one or more vascular endothelial cells may be carried out by administering to a patient in need thereof a therapeutically effective amount of an active agent as described herein. Where desired, the active agent may be administered using a composition as described herein. In particular embodiments, treatment of the one or more vascular endothelial cells with the active agent results in one or more of the following: preservation of vascular endothelial barrier function; promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cell. In one such embodiment, treatment of the one or more vascular endothelial cells with the active agent enhances the presence of VE-cadherin at the surface of vascular endothelial cells and promotes expression of p120-catenin at the surface of vascular endothelial cells. In another such embodiment, treatment of the one or more vascular endothelial cells with the active agent restores, at least in part, vascular barrier function after exposure of the vascular endothelial cells to one or more mediators of inflammation, wherein the one or more mediators of inflammation are selected from including one or more of an endotoxin (e.g., LPS), tumor necrosis factor (e.g., TNF-α), and IL-1β. The active agent may be selected from the active agents described herein and administration of the active agent may be accomplished by administration of such active agent using a composition as described herein.

The methods for promoting vascular endothelial barrier function described herein may be utilized for promoting barrier function in various different endothelial tissues, including endothelial tissues selected from one of vascular endothelium of the lung, vascular endothelium of the kidney and vascular endothelium of the spleen. Therefore, in specific embodiments, treating one or more endothelial cells with an active agent as described herein may include treating vascular endothelial cells selected from vascular endothelial cells of the lung, vascular endothelial cells of the kidney, and vascular endothelial cells of the spleen.

In another embodiment, the methods of the present invention include treating a patient at risk for or suffering from acute pulmonary vascular edema, chronic pulmonary vascular edema, acute pulmonary vascular inflammation, chronic pulmonary vascular inflammation, pulmonary fibrosis, including idiopathic pulmonary fibrosis, bacterial sepsis, or influenza infection, such as an avian flu infection. Therefore, in particular embodiments, the methods of the present invention include identifying a patient at risk of or suffering from one or more of acute pulmonary vascular edema, chronic pulmonary vascular edema, acute pulmonary vascular inflammation, chronic pulmonary vascular inflammation, pulmonary fibrosis, including idiopathic pulmonary fibrosis, bacterial sepsis, or influenza infection, such as an avian flu infection, and administering to the patient a therapeutically effective amount of an active agent as described herein. Where desired, the active agent may be administered using a composition as described herein. In specific embodiments, administration of the active agent results in one or more of the following: promotion of endothelial barrier function in the presence of one or more mediators of inflammation, including one or more of an endotoxin (e.g., LPS), tumor necrosis factor (e.g., TNF-α), and IL-1β; inhibition of vascular leak in the presence of one or more mediators of inflammation, including one or more of an endotoxin (e.g., LPS), tumor necrosis factor (e.g., TNF-α), and IL-1β; promotion of the presence of VE-cadherin at the surface of vascular endothelial cells; and promotion of expression of p120-catenin at the surface of vascular endothelial cells.

In another embodiment, the methods of the present invention include restoring vascular endothelial barrier function in a patient, wherein the patient is suffering from a pathologic condition selected from acute pulmonary vascular edema, chronic pulmonary vascular edema, acute pulmonary vascular inflammation, chronic pulmonary vascular inflammation, pulmonary fibrosis, including idiopathic pulmonary fibrosis, bacterial sepsis, or influenza infection, such as an avian flu infection. Moreover, the pathologic condition or environmental condition may be further associated with the presence or expression of one or more mediators of inflammation, such as for example, one or more of an endotoxin (e.g., LPS), tumor necrosis factor (e.g., TNF-α), and IL-1β. In a method for restoring vascular barrier function as described herein, a therapeutically effective amount of an active agent as described herein is administered to the patient. Where desired, the active agent may be administered using a composition as described herein. In specific embodiments, administration of the active agent results in one or more of the following: restoration of vascular barrier function; inhibition of vascular leak; enhanced presence of VE-cadherin at the surface of vascular endothelial cells; and enhanced expression of p120-catenin at the surface of vascular endothelial cells.

In yet further embodiments, the methods of the present invention include methods for promoting the presence of VE-cadherin at the surface of vascular endothelial cells. In particular embodiments, a method for promoting the presence of VE-cadherin at the surface of vascular endothelial cells includes treating one or more vascular endothelial cells with an active agent as described herein. In one such embodiment, the step of treating one or more vascular endothelial cells may be carried out by administering to a patient in need thereof a therapeutically effective amount of an active agent as described herein. Where desired, the active agent may be administered using a composition as described herein. In particular embodiments, treatment of the one or more vascular endothelial cells with the active agent results in one or both of promoting the presence of VE-cadherin at the surface of vascular endothelial cells and promoting expression of p120-catenin at the surface of vascular endothelial cells. In one such embodiment, treatment of the one or more vascular endothelial cells with the active agent enhances the presence of VE-cadherin at the surface of vascular endothelial cells and promotes expression of p120-catenin at the surface of vascular endothelial cells.

Methods of screening for or evaluating an agent that promotes vascular endothelial barrier function are also provided herein. For example, in particular embodiments, methods of screening for active agents according to the present description can be carried out using the in-vitro experiments and in-vivo models described herein. In a specific embodiment of a method of screening active agents as described herein, the method may include evaluating the ability of an active agent to promote the presence of VE-cadherin at the surface of vascular endothelial cells utilizing the experimental protocols provided herein. In another embodiment of a method of screening active agents as described herein, the method may include evaluating the ability of an active agent to promote expression of p120-catenin at the surface of vascular endothelial cells utilizing the experimental protocols provided herein. In yet a further embodiment, a method of screening active agents as described herein may include evaluating the ability of an active agent to preserve endothelial barrier function utilizing the experimental protocols provided herein. In still a further embodiment, a method of screening active agents may include evaluating the ability of an active agent to inhibit formation of pulmonary fibrosis in an animal model of Bleomycin-induced fibrosis, as described herein.

In a specific embodiment, a method for identifying an agent inhibits the activity or availability of a targeted ARF-GEF, such as a cytohesin, a cytohesin selected from the ARNO family of cytohesins, or ARNO, in a manner that results in inhibition the activity or availability of one or more ARFs, such as ARF6 and ARF1, involves an aptamer-displacement screen assay as described, for example, by Hafner et al. (Displacement of protein-bound aptamers with small molecules screened by fluorescence polarization, Nat Protoc (2008), 3, 579-587). In particular, such a method can be used to identify and confirm the activity of small molecules, such as those described herein. The association of the aptamer with its target is detected by fluorescence polarization. The fluorescence-labeled aptamer exhibits low polarization in the non-bound state. When bound to the target protein, the fluorescence polarization of the fluorescence-labeled aptamer is increased. If a small molecule displaces the aptamer from the protein, the fluorescence polarization of the fluorescence-labeled aptamer decreases, thereby allowing identification of small molecule candidates exhibiting activities analogous to the fluorescence labeled aptamer.

### IV. Examples

The Examples that follow are offered for illustrative purposes only and are not intended to limit the scope of the compositions and methods described herein in any way. It is to be understood that the disclosed compositions and methods are not limited to the particular methodologies, protocols, and reagents described herein. In each instance, unless otherwise specified, standard materials and methods were used in carrying out the work described in the Examples provided. All patent and literature references cited in the present specification are hereby incorporated by reference in their entirety.

The practice of the present invention employs, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, T., et al. (1982) Molecular Cloning: A Laboratory Manual (Cold Spring Harbor Laboratory, Cold Spring Harbor, N.Y.); Sambrook, J., et al. (1989) Molecular Cloning: A Laboratory Manual, 2^{nd} Ed. (Cold Spring Harbor Laboratory, Cold Spring Harbor, N.Y.); Ausubel, F. M., et al. (1992) Current Protocols in Molecular Biology, (J. Wiley and Sons, NY); Glover, D. (1985) DNA Cloning, I and II (Oxford Press); Anand, R. (1992) Techniques for the Analysis of Complex Genomes, (Academic Press); Guthrie, G. and Fink, G. R. (1991) Guide to Yeast Genetics and Molecular Biology (Academic Press); Harlow and Lane (1988) Antibodies: A Laboratory Manual (Cold Spring Harbor Laboratory, Cold Spring Harbor, N.Y.); Jakoby, W. B. and Pastan, I. H. (eds.) (1979) Cell Culture. Methods in Enzymology, Vol. 58 (Academic Press, Inc., Harcourt Brace Jovanovich (NY); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Hogan et al. (eds) (1994) Manipulating the Mouse Embryo; A Laboratory Manual, 2^{nd} Edition, Cold Spring Harbor Laboratory Press, Cold Spring Harbor, N.Y.). A general discussion of techniques and materials for human gene mapping, including mapping of human chromosome 1, is provided, e.g., in White and Lalouel (1988) Ann. Rev. Genet. 22:259 279. The practice of the present invention employs, unless otherwise indicated, conventional techniques of chemistry, molecular biology, microbiology, recombinant DNA, genetics, and immunology. (See, e.g., Maniatis et al., 1982; Sambrook et al., 1989; Ausubel et al., 1992; Glover, 1985; Anand, 1992; Guthrie and Fink, 1991).

Nothing herein is to be construed as an admission that the subject matter taught herein is not entitled to antedate such disclosure by virtue of prior invention. No admission is made that any reference constitutes prior art.

### Example 1

### Slit-Robo4 Signaling Reduces Endothelial Hyperpermeability Induced by Multiple Mediators of Inflammation

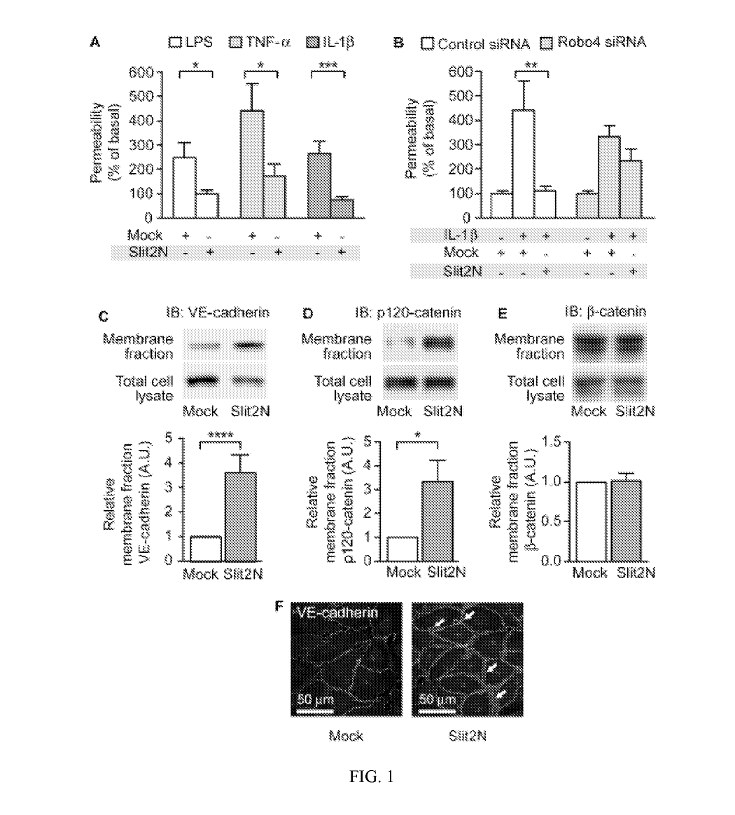

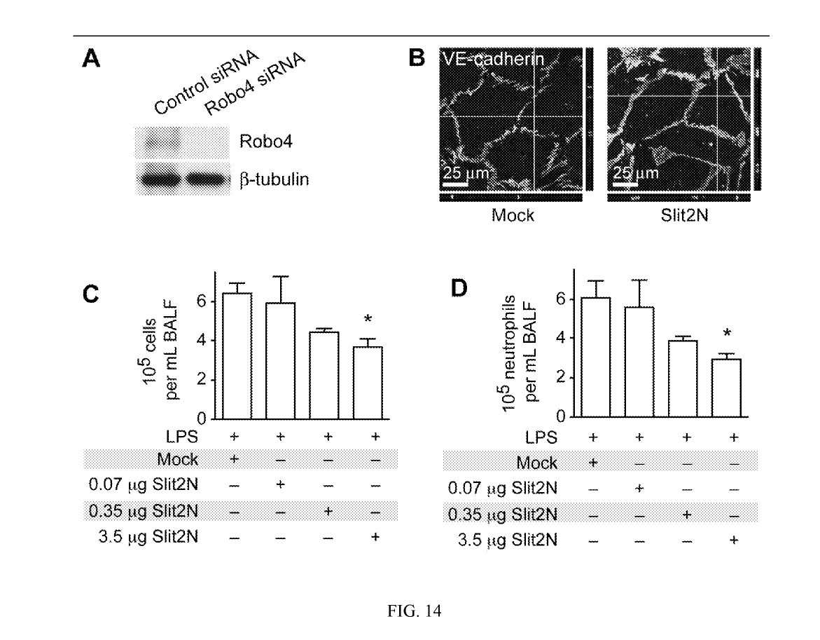

Slit-Robo4 signaling reduces endothelial hyperpermeability induced by endotoxin (lipopolysaccharide, LPS), tumor necrosis factor-α (TNF-α), and interleukin-1β (IL-1β), all important mediators of inflammation (Dinarello, C. A. 1997. Proinflammatory and anti-inflammatory cytokines as mediators in the pathogenesis of septic shock. Chest 112:321 S-329S). To study barrier function in vitro, we assessed the ability of a human endothelial cell monolayer to act as a barrier to diffusion of a horseradish peroxidase (HRP) reporter. We utilized the N-terminal fragment (Slit2N), which is the active fragment of Slit that is released by proteolytic cleavage (Chedotal, A. 2007. Slits and their receptors. Adv Exp Med Biol 621:65-80). As shown in FIG. 1a, Slit2N significantly reduced LPS, TNF-α, and IL-1β induced permeability. Furthermore, the inhibitory effect of Slit2N was lost in cells exposed to siRNA directed against Robo4 (FIG. 1B; FIG. 14A).

### Example 2

### Slit2-Robo4 Promotes Vascular Stability by Directly Enhancing the Machinery Responsible for Cell-Cell Interactions

The Slit2-Robo4 pathway promotes vascular stability by directly enhancing the machinery responsible for cell-cell interactions. In the endothelium, critical stabilizing interactions are mediated by the adherens junction protein, vascular endothelial cadherin (VE-cadherin) (Dejana, E., F. Orsenigo, and M. G. Lampugnani. 2008. The role of adherens junctions and VE-cadherin in the control of vascular permeability. J Cell Sci 121:2115-2122; and Vestweber, D. 2008. VE-cadherin: the major endothelial adhesion molecule controlling cellular junctions and blood vessel formation. Arterioscler Thromb Vasc Biol 28:223-232). We found that treating human microvascular lung endothelial cells (HMVEC-lung) with Slit2N significantly increased VE-cadherin levels at the cell surface junctions (FIG. 1C, F; FIG. 14B). VE-cadherin surface expression is regulated by the association of p120-catenin with VE-cadherin, an association known to inhibit VE-cadherin internalization from the cell surface and promote vascular stability (Potter, M. D., S. Barbero, and D. A. Cheresh. 2005. Tyrosine phosphorylation of VE-cadherin prevents binding of p120- and beta-catenin and maintains the cellular mesenchymal state. J Biol Chem 280:31906-31912; and Xiao, K., J. Garner, K. M. Buckley, P. A. Vincent, C. M. Chiasson, E. Dejana, V. Faundez, and A. P. Kowalczyk. 2005. p120-Catenin regulates clathrin-dependent endocytosis of VE-cadherin. Mol Biol Cell 16:5141-5151). Slit2N also increased cell surface p120-catenin expression (FIG. 1D), but had no observed effect on other junction or catenin family members (FIG. 1E, data not shown).

### Example 3

### Slit2 Enhances VE-Cadherin at the Cell Surface Following Exposure to IL-1β

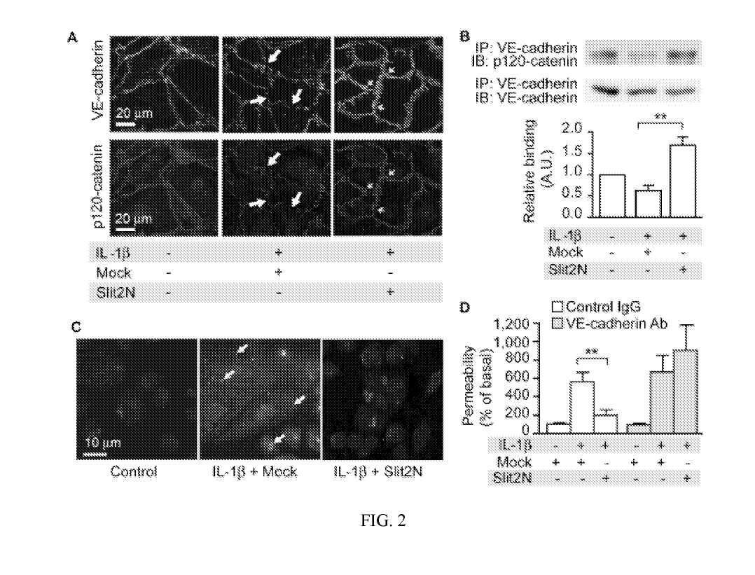

IL-1β reduces VE-cadherin levels at the cell surface and Slit2N negated this effect (FIG. 2A). IL-1β stimulation decreased p120-catenin at the cell surface and Slit2N reversed this effect (FIG. 2A). IL-1β-induced dissociation of VE-cadherin from p120-catenin and internalization of VE-cadherin (FIG. 2B, C). Slit2N restores association of VE-cadherin and p120-catenin, and blocks internalization of VE-cadherin (FIG. 2B, C). To investigate whether the effect of Slit2N on VE-cadherin localization is necessary for its ability to enhance vascular stability, we examined if an anti-VE-cadherin antibody could block the effect of Slit2N on permeability in vitro. Slit2N inhibited IL-1β-induced permeability in vitro in the presence of a non-specific IgG; however, the effect of Slit2N was lost in the presence of an anti-VE-cadherin antibody (FIG. 2D). Together, these data demonstrate that Slit preserves the association of p120-catenin with VE-cadherin in the face of IL-1β stimulation, thereby promoting vascular integrity by reducing cytokine induced VE-cadherin endocytosis.

### Example 4

### Slit2 Reduces Vascular Permeability In-Vivo Under Conditions of Cytokine Storm

To illustrate that Slit reduces vascular permeability in-vivo under conditions that result in cytokine storm, we utilized a bacterial endotoxin model of pulmonary inflammation. In this model, lipopolysaccharide (LPS) is administered to the lungs of mice through intratracheal instillation, simulating a gram-negative infection (Matute-Bello, G., C. W. Frevert, and T. R. Martin. 2008. Animal models of acute lung injury. Am J Physiol Lung Cell Mol Physiol 295:L379-399). LPS instillation in the lung is a model of acute inflammation. The administration of LPS triggers a massive inflammatory reaction and release of cytokines, resulting in a significant increase in alveolar capillary permeability.

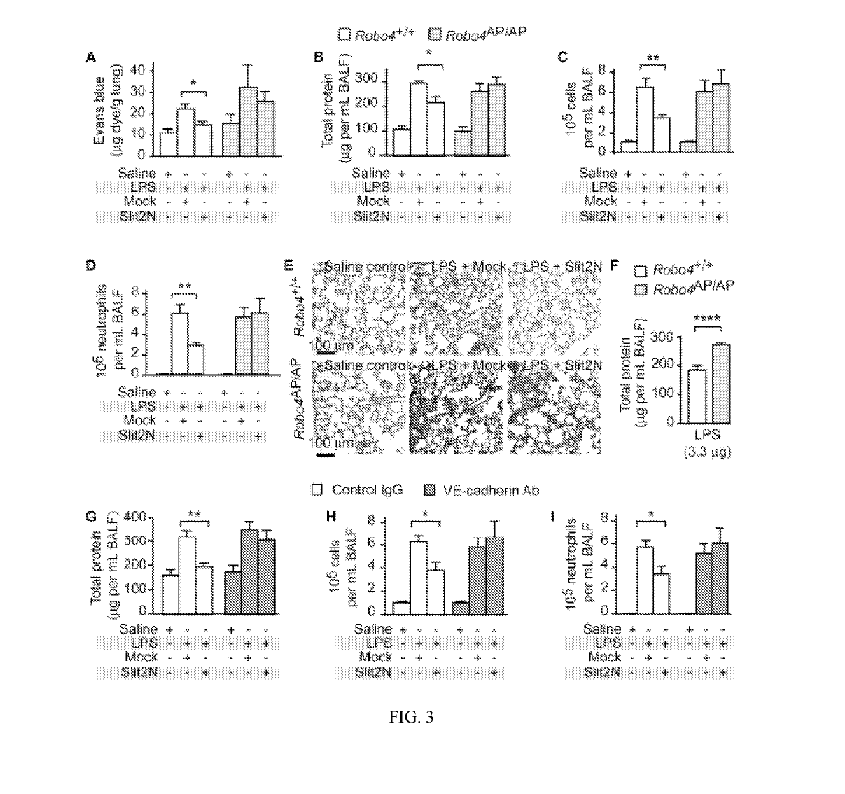

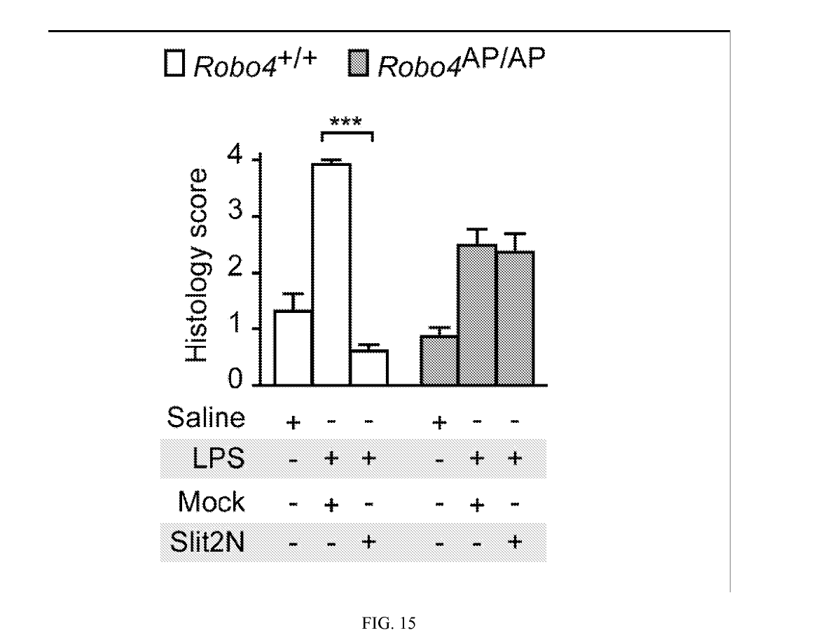

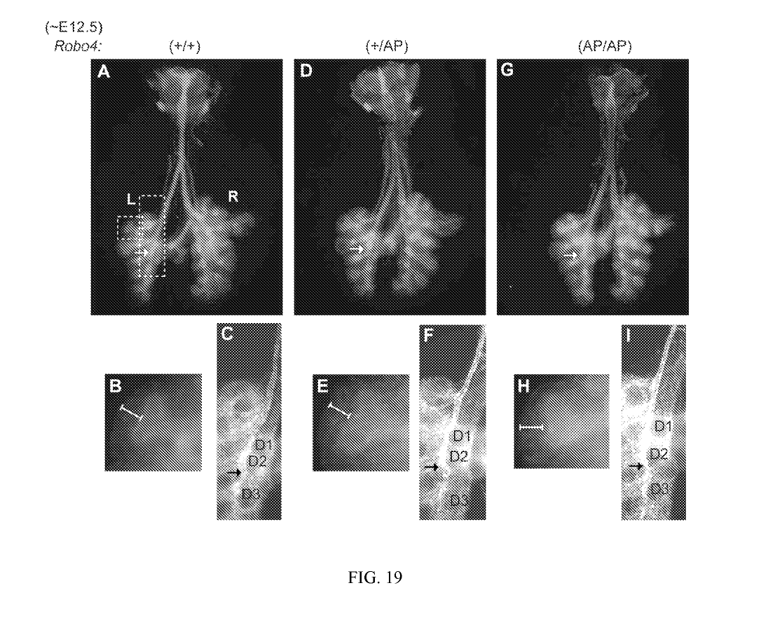

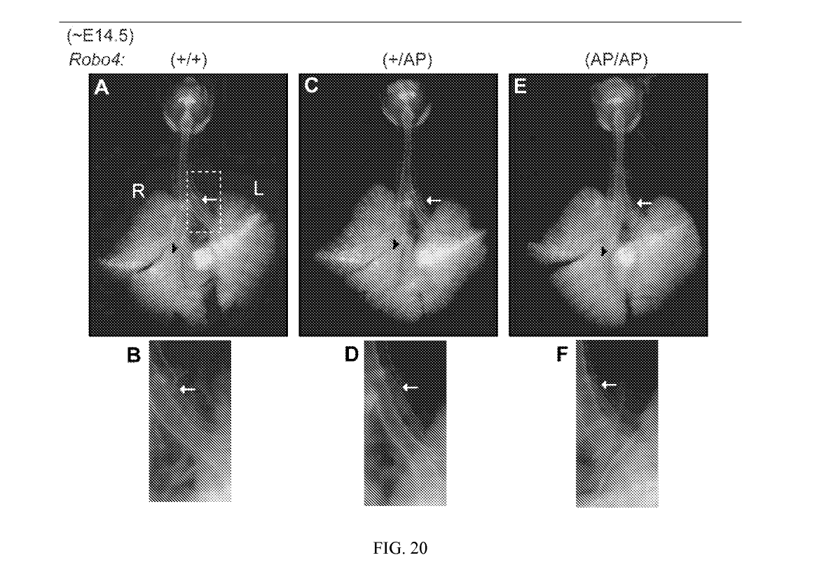

Using Evans Blue Albumin (EBA) as a tracer, we found that Slit2N significantly reduces vascular leak in the lungs of LPS treated Robo4^{+/+} mice (FIG. 3A). The effect of Slit2N was lost in Robo4-null (Robo4^{AP/AP}) mice, demonstrating that Robo4 is necessary for the effect of Slit2N in vivo (FIG. 3A). This result also indicates that this activity is endothelial-specific, as Robo4 is only detected in the endothelium (Huminiecki, L., M. Gorn, S. Suchting, R. Poulsom, and R. Bicknell. 2002. Magic roundabout is a new member of the roundabout receptor family that is endothelial specific and expressed at sites of active angiogenesis. Genomics 79:547-552; and Park, K. W., C. M. Morrison, L. K. Sorensen, C. A. Jones, Y. Rao, C. B. Chien, J. Y. Wu, L. D. Urness, and D. Y. Li. 2003. Robo4 is a vascular-specific receptor that inhibits endothelial migration. Dev Biol 261:251-267). LPS instillation in the lung also induces accumulation of protein exudates and leukocytes in the alveolar space, inflammatory responses that can be quantified by bronchoalveolar lavage (BAL) (Matute-Bello et al., 2008). Slit2N reduced protein exudates, a key marker of acute lung injury and indicator of vascular barrier disruption (Ware, L. B., and M. A. Matthay. 2000. The acute respiratory distress syndrome. N Engl J Med 342:1334-1349), and inflammatory cell accumulation in the bronchoalveolar lavage fluid (BALF) of Robo4^{+/+} mice in a dose-dependent manner (FIG. 3B-D; FIG. 14C, D). Inhibition of protein and leukocyte accumulation in BALF was lost in Robo4^{AP/AP} mice, indicating again that Slit2N acts directly upon the vasculature to decrease protein exudates and inflammatory cell accumulation in the alveoli (FIG. 3B-D). Finally, histological examination of the lung confirmed that Slit2N acts in a Robo4-dependent manner by reducing LPS-induced lung inflammation in Robo4^{+/+} but not Robo4^{AP/AP} mice (FIG. 3E; FIG. 15). We confirmed that the pulmonary vasculature of Robo4^{AP/AP} mice developed normally (FIGS. 19 and 20), indicating that the loss of the effect of Slit2 in Robo4^{AP/AP} was not due to structural differences in the vasculature.

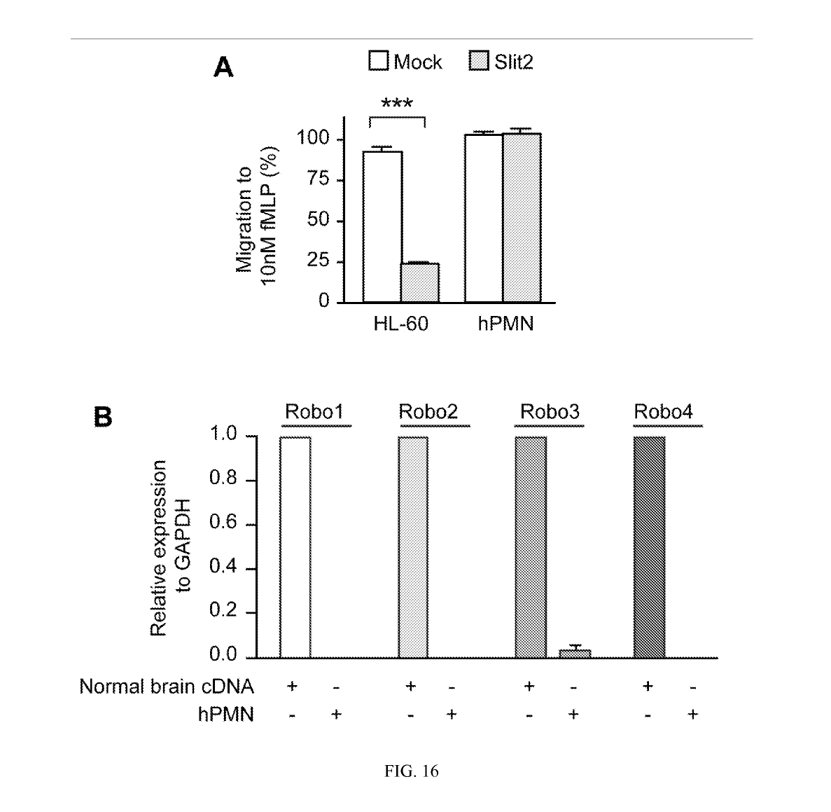

As neutrophils are a predominant cell type in bacterial pneumonia and LPS challenge models (Matute-Bello et al., 2008), we asked if Slit2N had a direct effect on neutrophil migration. It has previously been reported that Slit2 has a profound impact on neutrophil migration (Wu, J., et al. The neuronal repellent Slit inhibits leukocyte chemotaxis induced by chemotactic factors. Nature 410, 948-952 (2001). However, these experiments were done using DMSO-treated HL-60 neutrophil-like cells. We observed the same effect of Slit2 on DMSO-treated HL-60 neutrophil-like cells, but found primary human PMNs did not respond to Slit2N, which is consistent with the fact that they do not express Robo receptors (FIG. 16A, B).

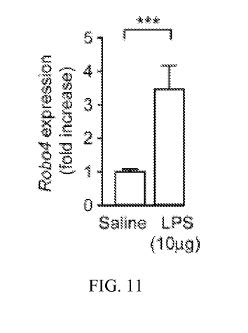

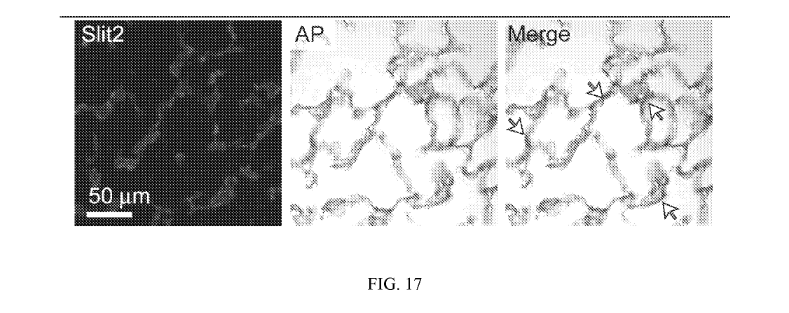

In initial studies, we did not detect enhanced sensitivity in the lungs of Robo4^{AP/AP} mice compared to Robo4^{+/+} mice in the LPS-model of acute inflammation (FIG. 3B-D). However, using quantitative PCR, we found that Robo4 expression was significantly increased in the lung 6 hours after LPS instillation (FIG. 11). This result suggested that Robo4 may be important for inhibiting or resolving LPS-induced inflammation in the lung. We reasoned that if the level of LPS administered was too high, it would cause such severe damage that any difference between the two genotypes would be masked. Thus, we lowered the dose of LPS used to challenge the mice, and found that Robo4^{AP/AP} mice had significantly higher protein levels in BALF exudates compared to littermate Robo4^{AP/AP} mice (FIG. 3F). This increased sensitivity indicates a role for endogenous Slit-Robo4 signaling in dampening the vascular effects of cytokines Consistent with this model, Slit2 protein is expressed throughout the lung and in close proximity to the endothelium (FIG. 17).

### Example 5

### Slit Signals Via a VE-Cadherin Dependent Mechanism In-Vivo

To confirm that Slit signals via a VE-cadherin dependent mechanism in-vivo, we blocked VE-cadherin with a specific antibody that prevents homophilic interactions between VE-cadherin expressed on adjacent endothelial cells. Slit2N reduced protein exudates and inflammatory cell infiltration in the presence of a control IgG antibody, but not in the presence of a VE-cadherin blocking antibody (FIG. 3G-I). Thus, similar to the results of cell culture experiments, the in-vivo data support a model of Slit-Robo4 promoting VE-cadherin expression at the cell surface and blunting of cytokine mediated endothelial hyperpermeability.

### Example 6

### Various Slit Proteins Work to Activate Robo4

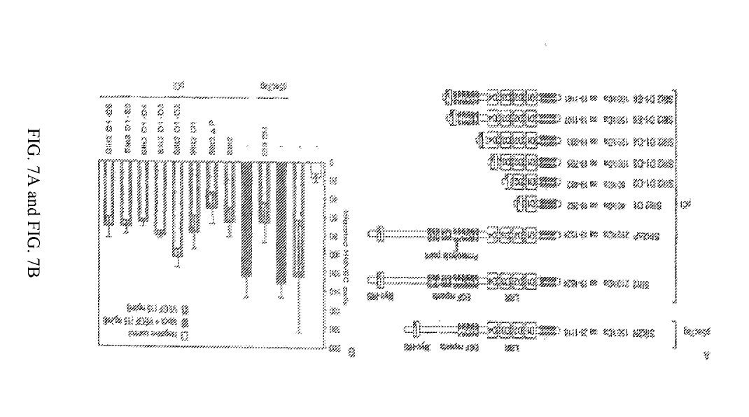

FIG. 7 illustrates various constructs of the Slit2 protein. As has already been described herein, the 150 kD protein Slit2N (SEQ ID NO: 4), has been found to be effective in in vitro and in vivo models, including Miles assays, assays for retinal permeability, tube formation and endothelial cell migration and in OIR and CNV models of ocular disease. In FIG. 7A, different constructs for the Slit protein are depicted. The four leucine rich domains (LRR), the epidermal growth factor homology region (EGF) and the c-terminal tags (MYC/HIS) are indicated. Inhibition of VEGF mediated endothelial cell migration by the different Slit constructs (2 nM) is shown in FIG. 7B.

### Example 7

### Robo4 Knockout Mouse

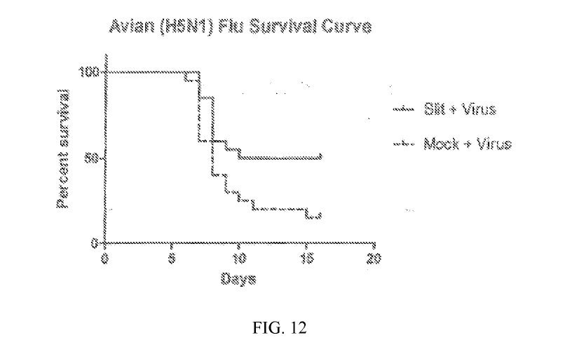

The Robo4 knockout mice utilized in the experimental examples detailed herein were produced using standard techniques. To produce the knockout mice, exons one through five of the gene expressing Robo4 were replaced with an alkaline phosphatase (AP) reporter gene using homologous recombination. This allele, Robo4^{AP}, lacked the exons encoding the immunoglobulin (IgG) repeats of the Robo4 ectodomain, which are predicted to be required for interaction with Slit proteins. The Robo4^{+/AP} animals were intercrossed to generate mice that were homozygous for the targeted allele. An illustration of the genomic structure of the mice is provided in FIG. 12. Robo4^{AP/AP} animals were viable and fertile, and exhibited normal patterning of the vascular system. These data indicate that Robo4 is not required for sprouting angiogenesis in the developing mouse, and point to an alternate function for Robo4 signaling in the mammalian endothelium. Alkaline phosphatase activity was detected in these animals throughout the endothelium of all vascular beds in the developing embryos and in the adult mice, which confirmed that the Robo4^{AP} allele is a valid marker of Robo4 expression.

### Example 8

### Slit2 Reduces Vascular Permeability and Development of Pulmonary Fibrosis In-Vivo

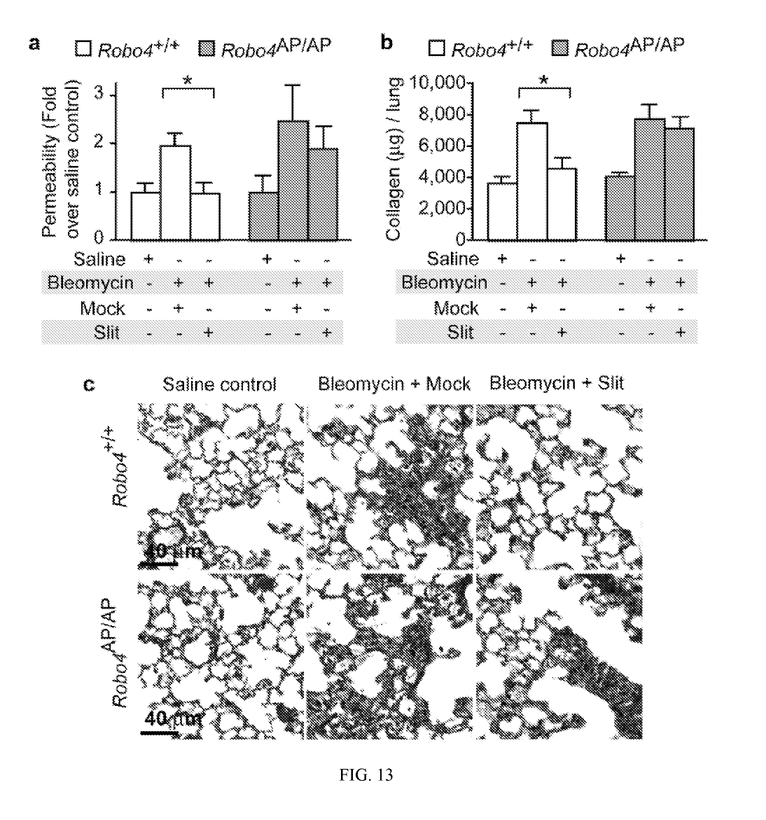

Slit2N reduces vascular permeability in-vivo in the setting of chronic inflammation (Matute-Bello, G., Frevert, C., & Martin, T. Animal models of acute lung injury. Am J Physiol Lung Cell Mol Physiol 295, 379-399 (2008); Gasse, P., et al. IL-1R1/MyD88 signaling and the inflammasome are essential in pulmonary inflammation and fibrosis in mice. J Clin Invest 117, 3786-3799 (2007); Russo, R., et al. Role of the chemokine receptor CXCR2 in bleomycin-induced pulmonary inflammation and fibrosis. Am J Respir Cell Mol Biol (2008)). Bleomycin is a chemical that causes prolonged and chronic permeability in the lung (Tager, A., et al. The lysophosphatidic acid receptor LPA1 links pulmonary fibrosis to lung injury by mediating fibroblast recruitment and vascular leak. Nat Med 14, 45-54 (2008)). In addition to chronic permeability and inflammation, Bleomycin causes pulmonary fibrosis. In Robo4^{+/+} mice, Slit2N significantly reduced Bleomycin-induced EBA accumulation in the lung eleven days after Bleomycin administration (FIG. 13a). The effect of Slit2N was lost in Robo4^{AP/AP} mice (FIG. 13a). Slit2N also significantly reduced Bleomycin-induced pulmonary fibrosis (FIG. 13b). This effect was lost in Robo4^{AP/AP} mice, indicating that Slit2N acted directly upon the endothelium to reduce pulmonary fibrosis (FIG. 13b). Histologic examination of the lung using a trichrome stain to enhance the visualization of collagen deposition confirmed the effect of Slit2N in a Robo4-dependent manner (FIG. 13c). These results highlight the value in of vascular stabilization in reducing pulmonary fibrosis and underscore the role the endothelium plays in the pathogenesis of fibrosis.

### Example 9

### SecinH3 Inhibits Bleomycin-Induced Fibrosis In-Vivo

The ability of SecinH3 to inhibit pulmonary fibrosis was evaluated in an animal model of Bleomycin-induced fibrosis, as described in Example 8. In brief, 6-8 week old mice were anesthetized and given Bleomycin (0.05 U in 40 μL saline) by intranasal instillation. Control mice received an intranasal instillation of 40 μL saline. Mice were given an intraperitoneal injection twice daily of 30 uM SecinH3 or vehicle. On day 11, mice were sacrificed by CO₂ asphyxiation, lungs removed, and homogenized in 0.5M acetic acid with protease inhibitors (Roche). Pulmonary collagen content was assessed using the Sircol collagen assay. Homogenates were incubated overnight at 4° C. with stirring. Samples were then centrifuged and 1 mL Sircol dye reagent was added to 100 μL of supernatant for 30 minutes. Samples were again centrifuged, the pellet re-suspended with 1 mL alkali reagent, and analyzed by spectrophotometry (Biocolor). These samples were compared against a collagen standard curve provided by the manufacturer. Data are presented as s.e.m. of at least seven mice per condition.

### Example 10

### Slit Enhances Vascular Stability During Polymicrobial Sepsis

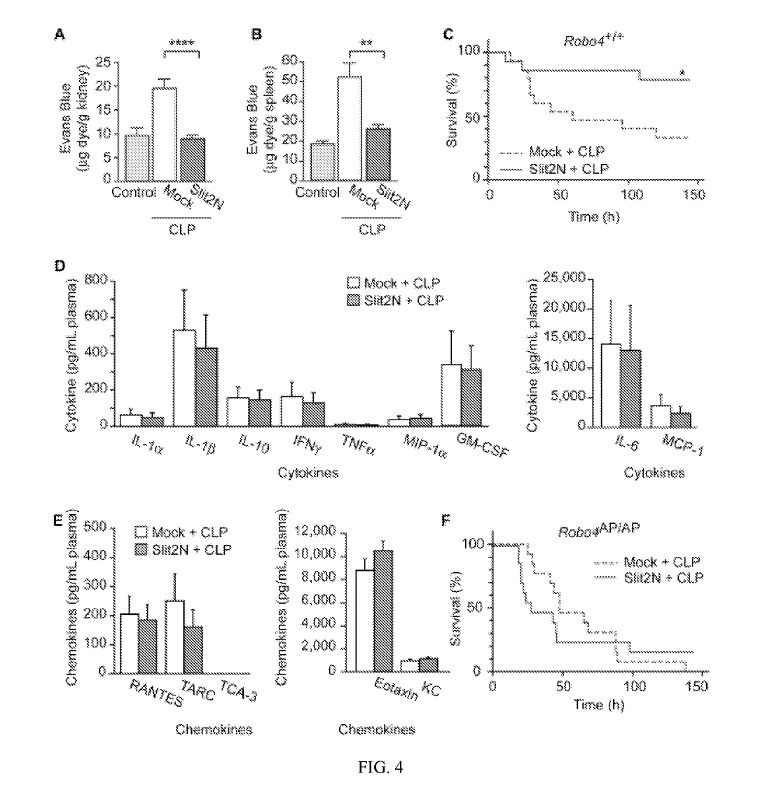

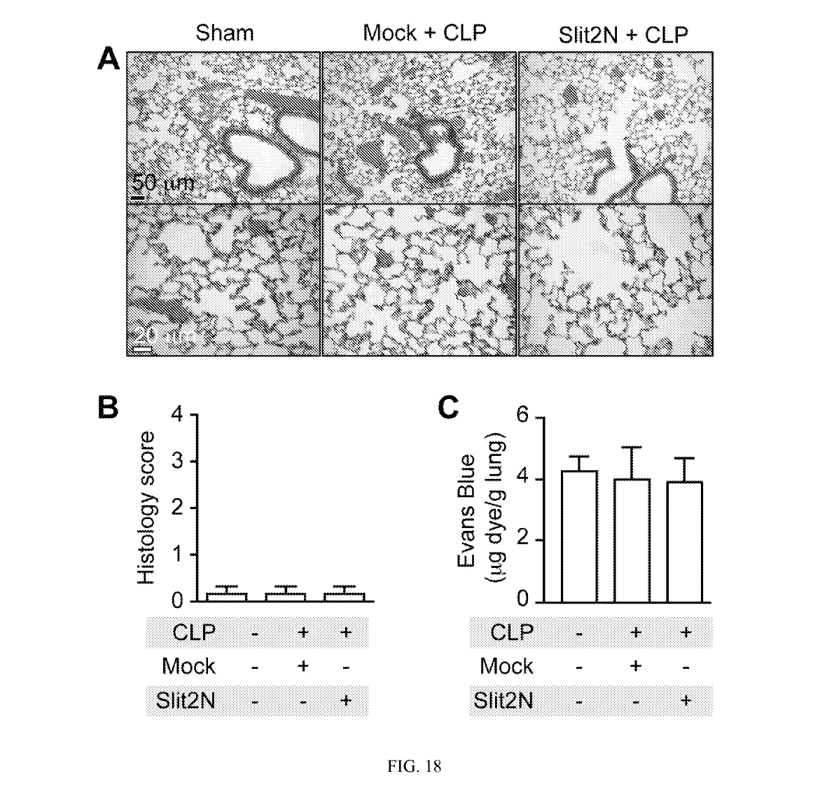

Slit2N reduces mortality in the setting of systemic vascular instability, and the effects of Slit2N are not limited to the lung. In a model of polymicrobial sepsis known as cecal ligation and puncture (CLP) (Hubbard, W. J., M. Choudhry, M. G. Schwacha, J. D. Kerby, L. W. Rue, 3rd, K. I. Bland, and I. H. Chaudry. 2005. Cecal ligation and puncture. Shock 24 Suppl 1:52-57). Slit2N significantly reduced vascular permeability in the kidney and spleen (FIG. 4A, B) and improved the survival of mice exposed to CLP-induced sepsis from 33% to nearly 80% (FIG. 4C). Under the conditions of these experiments, CLP did not cause significant damage to the lung, providing further support that the effect of Slit2N was not limited to the lung (FIG. 18A-C). Because a hyper-inflammatory response contributes to the pathogenesis of sepsis, we tested whether Slit2N affects cytokine and chemokine levels in the plasma of septic mice (Dinarello, C. A. 1997. Proinflammatory and anti-inflammatory cytokines as mediators in the pathogenesis of septic shock. Chest 112:321 S-329S). Slit2N did not alter the expression of a panel of cytokines and chemokines, demonstrating that the therapeutic effect of Slit2N is not secondary to a reduction in inflammatory cytokine and chemokine levels (FIG. 4D, E). Finally, the effect of Slit2N was lost in Robo4^{AP/AP} mice (FIG. 4F), demonstrating that Robo4 is necessary for the activity of Slit2N. Taken together, these data demonstrate that Slit enhances survival during the systemic inflammatory response triggered by sepsis by specifically enhancing vascular stability.

### Example 11

### Slit Enhances Vascular Stability Resulting from Viral Infection

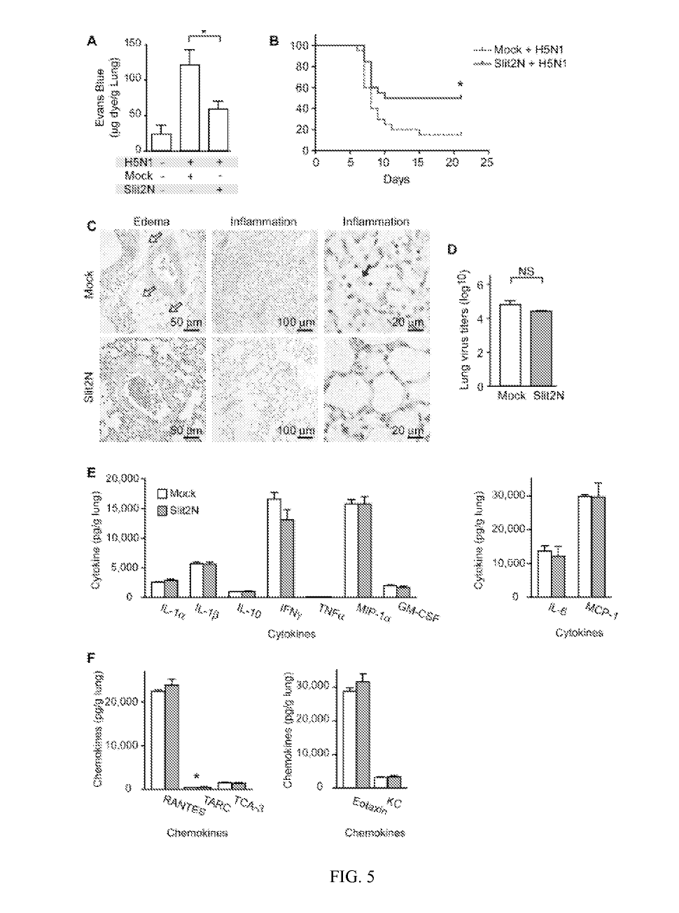

The effects of Slit were examined in a model of H5N1 influenza. Pandemic influenzas such as avian flu (H5N1) are examples of lung injury caused by direct infection that are often characterized by a massive increase in cytokine levels and excessive inflammation (de Jong, M. D., C. P. Simmons, T. T. Thanh, V. M. Hien, G. J. Smith, T. N. Chau, D. M. Hoang, N. V. Chau, T. H. Khanh, V. C. Dong, P. T. Qui, B. V. Cam, Q. Ha do, Y. Guan, J. S. Peiris, N. T. Chinh, T. T. Hien, and J. Farrar. 2006. Fatal outcome of human influenza A (H5N1) is associated with high viral load and hypercytokinemia. Nat Med 12:1203-1207; Kobasa, D., S. M. Jones, K. Shinya, J. C. Kash, J. Copps, H. Ebihara, Y. Hatta, J. H. Kim, P. Halfmann, M. Hatta, F. Feldmann, J. B. Alimonti, L. Fernando, Y. Li, M. G. Katze, H. Feldmann, and Y. Kawaoka. 2007; Aberrant innate immune response in lethal infection of macaques with the 1918 influenza virus. Nature 445:319-323; and Abdel-Ghafar, A. N., T. Chotpitayasunondh, Z. Gao, F. G. Hayden, D. H. Nguyen, M. D. de Jong, A. Naghdaliyev, J. S. Peiris, N. Shindo, S. Soeroso, and T. M. Uyeki. 2008. Update on avian influenza A (H5N1) virus infection in humans. N Engl J Med 358:261-273). Slit2N significantly inhibited endothelial hyperpermeability in the lung three days post-H5N1 infection (FIG. 5A) and reduced mortality (FIG. 5B). The lung pathology in Slit2N treated mice was decreased in severity compared to mock treated mice (FIG. 5C). To exclude the possibility that Slit2N has a direct anti-viral activity, we measured lung viral titers and found that Slit2N did not alter viral load (FIG. 5D); further, Slit2N does not significantly reduce the level of inflammatory cytokine release following H5N1 infection (FIG. 5E, F). Thus, the H5N1 results are consistent with our LPS and CLP studies, and indicate that specifically limiting the vascular response to hypercytokinemia is sufficient to reduce mortality and morbidity in animal models of serious infections.

### V. Materials & Methods

Preparation of recombinant Slit2N. 293T cells plated onto Poly-L-lysine (Sigma) coated dishes were transiently transfected with empty vector pSecTagB or pSecTagB::hSlit2N. For each 15 cm dish of cells, 60 μg DNA and 100 μL Lipofectamine (Invitrogen) in serum-free Opti-MEM was used. Slit2N protein was salt extracted as previously described in Jones, C. A., N. R. London, H. Chen, K. W. Park, D. Sauvaget, R. A. Stockton, J. D. Wythe, W. Suh, F. Larrieu-Lahargue, Y. S. Mukouyama, P. Lindblom, P. Seth, A. Frias, N. Nishiya, M. H. Ginsberg, H. Gerhardt, K. Zhang, and D. Y. Li. 2008. Robo4 stabilizes the vascular network by inhibiting pathologic angiogenesis and endothelial hyperpermeability. Nat Med 14:448-453. Using this protocol, Slit2N concentrations of 0.5-1.5 mg/mL were routinely obtained. We performed the same salt extraction procedure on cells transfected with empty vector pSecTagB. This preparation is referred to as Mock and was used as a control for Slit2N in all experiments. In vitro studies were conducted using 10 nM Slit2N.

LPS-induced acute lung injury. Eight to twelve week old C57BL/6 mice were injected intravenously (IV) with saline alone or 3.5 μg Slit2N or Mock in saline. Alternatively, the intravenous injection also contained 20 μg control IgG or 20 μg VE-cadherin blocking antibody (clone BV13, eBiosciences). Animals were anesthetized with Avertin before surgical exposure of the trachea. 10 μg Lipopolysaccharide (serotype 0111:B4, Sigma) in 100 μL saline or saline alone was administered intratracheally (IT). Twenty four hours later, the trachea was re-exposed and catheterized. Bronchoalveolar lavage fluid (BALF) was obtained by injection of 1 mL saline followed by aspiration repeated three times. BALF was centrifuged at 300 g for 5 minutes to recover inflammatory cells. The pellet was treated with ACK buffer for 3 minutes to remove red blood cells. Cells were centrifuged at 300 g for 5 minutes, and resuspended in 1 mL PBS containing 1% FBS. Cell counts were then determined by hemocytometer. Neutrophil counts were determined by cell differential counts. BALF protein was assessed by protein assay (BioRad). Data are presented as s.e.m. of at least five mice per condition.

Bleomycin model. Bleomycin experiments were carried out as described in (Gasse, P., et al. IL-1R1/MyD88 signaling and the inflammasome are essential in pulmonary inflammation and fibrosis in mice. J Clin Invest 117, 3786-3799 (2007)). In brief, 6-8 week old mice were anesthetized and given Bleomycin (0.05 U in 40 μL, saline) by intranasal instillation. Control mice received an intranasal instillation of 40 μL saline. Mice were given a daily intraperitoneal injection of 5 μg Slit or Mock. Control mice received a daily injection of saline. On day 11, mice were sacrificed by CO₂ asphyxiation, lungs removed, and homogenized in 0.5M acetic acid with protease inhibitors (Roche). Homogenates were incubated overnight at 4° C. with stirring. Samples were then centrifuged and 1 mL Sircol dye reagent was added to 100 μL of supernatant for 30 minutes. Samples were again centrifuged, the pellet resuspended with 1 mL alkali reagent, and analyzed by spectrophotometry (Biocolor). These samples were compared against a collagen standard curve provided by the manufacturer. Data are presented as s.e.m. of at least five mice per condition.

H5N1 infection. Female 18-20 g BALB/c mice (Charles River Laboratories) were anesthetized and infected with H5N1 virus (Influenza A, Duck/MN/1525/81) by intranasal instillation. Mice were given an intravenous injection of 1.56 μg Slit or Mock daily for 5 days. Survival rate of mice subjected to H5N1 lung infection was determined for 21 days with 20 mice per condition.

Cecal ligation and puncture (CLP) sepsis model. Seven to eleven week old male C57BL/6 mice were given an intraperitoneal injection of 5 μg Slit2N or Mock. One hour later, mice were anesthetized with isoflurane, and CLP performed as described in Gomes, R. N., R. T. Figueiredo, F. A. Bozza, P. Pacheco, R. T. Amancio, A. P. Laranjeira, H. C. Castro-Faria-Neto, P. T. Bozza, and M. T. Bozza. 2006. Increased susceptibility to septic and endotoxic shock in monocyte chemoattractant protein 1/cc chemokine ligand 2-deficient mice correlates with reduced interleukin 10 and enhanced macrophage migration inhibitory factor production. Shock 26:457-463. Mice continued to receive an intraperitoneal injection of 5 μg Slit2N or Mock once a day. Mice in the sham operation group were subjected to identical procedures, except that ligation and puncture of the cecum were omitted. Survival rate of mice subjected to CLP was determined for 6 days with N=14 for Slit2N treatment and N=15 for Mock treatment for Robo4^{+/+} mice. For Robo4^{AP/AP} mice, N=13 for Slit2N treatment and N=13 for Mock treatment.

Evans Blue permeability. Vascular permeability in the lung was assessed using Evans Blue Albumin (EBA) as described in Moitra et al. (Moitra, J., S. Sammani, and J. G. Garcia. 2007. Re-evaluation of Evans Blue dye as a marker of albumin clearance in murine models of acute lung injury. Transl Res 150:253-265). Five hours after IT instillation of LPS, 4 hours after CLP, and 3 days after H5N1 infection, mice were given an IV injection of EBA (20 mg/kg). EBA was allowed to circulate for 1 hour, mice were deeply anesthetized, and perfused with saline+5 mM EDTA. Lungs were excised, weighed, and homogenized in 2 mL PBS. Four mL of formamide (Invitrogen) was added and the samples were incubated overnight at 60° C. to extract Evans Blue dye. The samples were then centrifuged and supernatants analyzed by spectrophotometry at both 620 and 740 nm. For CLP treated mice, mice were perfused, the kidneys and spleen were removed, weighed, and placed in formamide for 48 hours at 60° C. The absorbances were normalized as described in Moitra et al. and converted to μg Evans Blue dye per gram wet weight of lungs, kidneys or spleen respectively. Data are presented as s.e.m. of at least four mice per condition.

Histology. Twenty-four hours after LPS exposure or CLP, mice were euthanized by CO₂ asphyxiation. Chest cavities were opened, and lungs were inflated with ZnSO₂ buffered 10% Formalin. Formalin fixed tissues were processed routinely, embedded in paraffin, sectioned at 6 microns, and stained with H&E. Histologic quantification was modified from methods described in Gupta et al. (Gupta, N., X. Su, B. Popov, J. W. Lee, V. Serikov, and M. A. Matthay. 2007. Intrapulmonary delivery of bone marrow-derived mesenchymal stem cells improves survival and attenuates endotoxin-induced acute lung injury in mice. J Immunol 179:1855-1863). For H5N1 samples, six days post-infection the right lobes of the lungs from two animals were harvested and fixed in 10% neutral buffered formalin. Formalin fixed tissues were processed routinely, embedded in paraffin, sectioned at 5 microns, stained with H&E, and evaluated for microscopic lesions by a board certified veterinary pathologist.

RNA silencing of Robo4. For each experiment, HMVEC-L, P-4 (Lonza) were grown until 70-80% confluent in EGM-2mv medium (Lonza) in a 150 cm flask. Prior to trypsinizing, 12 Fibronectin-coated 6.5 mm 3.0 μm pore Transwells (Costar) received 25 μl/well of a huRobo4siRNA duplex (Qiagen, Hs_ROBO4_{—}1_HP, #1919431) diluted to 480 nM in RNA Suspension Buffer (Qiagen), and a second set of 12 transwells received 25 μl/well of an equimolar AllStars NegativeControl siRNA (Qiagen, #1027280). To form transfection complexes, siRNAs were premixed for 10 minutes at room temperature in the Transwells with 25 μl/well of HiPerfect Transfection Reagent (Qiagen), diluted 1:10 in OptiMEM (Invitrogen). Cells were lifted, and 150 μl complete medium, containing 2×10⁴ cells/well were seeded onto the transfection complexes. Transfectants were incubated for 30 minutes at 37° C., and 800 μl complete medium was added to the lower chamber of each Transwell. The transfected monolayers were cultured 48 h further, before performing the In Vitro permeability assay, as described. To confirm ablation of the Robo4 gene product, two 100 mm dishes were seeded with the remaining HMVEC-L, at 10⁶ cells/dish in 4.5 ml EGM-2mv, receiving either Robo4si RNA or AllStars Negative Control transfection complexes consisting of 12.5 μl of 20 μmRNA+70 μl HiPerfect+500 μl Opti MEM, pipetted on top of each dish of cell suspension. After 30 minutes at 37° C., 6.5 ml complete media, was added, and the plates were incubated for 48 h, followed by harvesting of lysate and Western blotting with 1:100 anti-Robo4 (N-17) and 1:200 anti-βTubullin antibodies (Santa Cruz Biotechnology).

Robo4 siRNA knockdown. huRobo4 siRNA duplex (Hs_ROBO4_{—}1_HP #1919431, Qiagen) or equimolar AllStars Negative Control siRNA (#1027280, Qiagen) transfection complexes were formed according to standard protocol and added to the upper chamber of Transwell filters. Twenty four hours later, cells were transfected a second time with huRobo4 or Control siRNA. After an additional 24 hours, in vitro permeability was assessed as previously described. Data are presented as mean±s.e.m. of at least three independent experiments performed in triplicate. Successful knockdown of Robo4 protein expression was confirmed by Western Blot using antibodies against Robo4 (N-17) or β-Tubulin (Santa Cruz Biotechnology).

In Vitro Permeability Assay. In vitro permeability was performed as described in Jones et al. (Jones, C. A., N. R. London, H. Chen, K. W. Park, D. Sauvaget, R. A. Stockton, J. D. Wythe, W. Suh, F. Larrieu-Lahargue, Y. S. Mukouyama, P. Lindblom, P. Seth, A. Frias, N. Nishiya, M. H. Ginsberg, H. Gerhardt, K. Zhang, and D. Y. Li. 2008. Robo4 stabilizes the vascular network by inhibiting pathologic angiogenesis and endothelial hyperpermeability. Nat Med 14:448-453). The assay was conducted using 100 ng/mL lipopolysaccharide (serotype 0111:B4, Sigma) for 3 hours, 10 ng/mL Tumor Necrosis Factor-alpha (TNF-α, R&D Systems) for 6 hours, or 10 ng/mL Interleukin-1 beta (IL-1β, R&D Systems) for 2 hours. As indicated, this was repeated in the presence of 25 μg/mL control rabbit IgG (Jackson ImmunoResearch) or 25 μg/mL anti-human VE-cadherin antibody (RDI Fitzgerald). Basal permeability for unstimulated monolayers was set at 100%. Data are presented as mean±s.e.m of at least three independent experiments performed in triplicate.

Subcellular fractionation. HMVEC-lung were treated with Slit2N or Mock in 0.1% FBS EBM-2 for 1.5 hours. Cells were then washed twice with ice-cold PBS containing Ca²⁺/Mg²⁺, once with HLB buffer (10 mM Tris-HCl PH7.4, 5 mM KCl, 1 mM MgCl₂), and collected in HLB buffer supplemented with protease inhibitors (Roche), phosphatase inhibitors (Sigma) and 1 mM DTT. Cells were then dounce-homogenized (20 strokes). The homogenate was centrifuged at 400 g for 10 minutes at 4° C. to pellet cell debris. The resulting supernatant was centrifuged again at 16,000 g for 30 minutes at 4° C. The pellet was washed once with HLB and resuspended in RIPA buffer for 30 minutes at 4° C. The resuspended pellet was centrifuged (16,000 g/15 min at 4° C.), and the resulting supernatant was saved as soluble membrane fraction. To obtain the total cell lysate an aliquot was saved prior to dounce homogenization. RIPA buffer was added to this aliquot and centrifuged at 13,000 g for 10 minutes at 4° C. The supernatant was saved and used as total cell lysate. Antibodies to VE-cadherin were obtained from Cell Signaling, and p120-catenin and β-catenin from BD biosciences. Densitometry was performed on at least three independent experiments and data are presented as mean±s.e.m.

Immunofluorescence. Immunofluorescence was performed as described in Jones, C. A., N. R. London, H. Chen, K. W. Park, D. Sauvaget, R. A. Stockton, J. D. Wythe, W. Suh, F. Larrieu-Lahargue, Y. S. Mukouyama, P. Lindblom, P. Seth, A. Frias, N. Nishiya, M. H. Ginsberg, H. Gerhardt, K. Zhang, and D. Y. Li. 2008. Cells were pre-treated with Slit2N or Mock for 30 minutes followed by stimulation with 10 ng/mL IL-1β for 3 hours. Primary antibodies to VE-cadherin (BD biosciences) or p120-catenin (Santa Cruz) were applied at 4° C. overnight. Images are representative of three independent experiments.

Immunoprecipitation. HMVEC-lung were treated with Slit2N or Mock in 0.1% FBS EBM-2 for 30 minutes. Cells were then stimulated with 10 ng/mL IL-1β for 10 minutes. HMVEC-lung were then washed with ice-cold PBS and lysed with ice-cold lysis buffer (10 mM Tris-HCl pH7.4, 50 mM NaCl, 1% NP-40 and 10% glycerol) supplemented with protease inhibitors, phosphatase inhibitors and 1 mM DTT. Cell lysates were incubated on ice for 30 min and centrifuged at 13,000 g for 15 minutes to pellet cell debris. Protein concentrations were determined by BCA assay (PIERCE) and 0.5 mg lysate were incubated with 8 μg of VE-cadherin antibody (Cell Signaling) and protein A/G sepharose (Santa Cruz) for 1 hour at 4° C. Complexes were washed three times with lysis buffer. The immunoprecipitates were subjected to Western blot analysis. Densitometry was performed on three independent experiments and data are presented as mean±s.e.m.

Internalization assay. VE-cadherin internalization was performed as described Xiao, K., J. Garner, K. M. Buckley, P. A. Vincent, C. M. Chiasson, E. Dejana, V. Faundez, and A. P. Kowalczyk. 2005. p120-Catenin regulates clathrin-dependent endocytosis of VE-cadherin. Mol Biol Cell 16:5141-5151. In brief, HMVEC-lung were seeded onto chamber slides and cultured for 72 hours. The media was then removed and the cells labeled for 30 minutes at 4° C. with anti-VE-cadherin antibody (clone BV6, RDI Fitzgerald). Cells were then pre-treated with Slit2N or Mock for 30 minutes. Excess antibody was removed by washing twice on ice with ice-cold media. Chamber slides were moved to 37° C. and incubated for 1 hour with 10 ng/ml IL-1β and 0.6 mM primaquine in the presence of 10 nM Slit2N or Mock. Cells were acid-washed to strip the surface-bound VE-cadherin. Monolayers were washed, fixed, and permeablized. Internalized VE-cadherin antibody was detected with Alexa 488-conjugated donkey anti-mouse IgG (Molecular Probes). Images are representative of four independent experiments.

Lung immunofluorescence. Adult Robo4^{+/AP} mice were euthanized by CO₂ asphyxiation. Chest cavities were opened, lungs inflated with OCT, and frozen quickly in OCT on dry ice. Lung sections were stained for alkaline phosphatase activity denoting areas of Robo4 expression as described in Jones, C. A., N. R. London, H. Chen, K. W. Park, D. Sauvaget, R. A. Stockton, J. D. Wythe, W. Suh, F. Larrieu-Lahargue, Y. S. Mukouyama, P. Lindblom, P. Seth, A. Frias, N. Nishiya, M. H. Ginsberg, H. Gerhardt, K. Zhang, and D. Y. Li. 2008. Robo4 stabilizes the vascular network by inhibiting pathologic angiogenesis and endothelial hyperpermeability. Nat Med 14:448-453. Sections were then stained using primary antibodies against Slit2 (E-20, Santa Cruz) or CD-31 (Pharmingen) followed by fluorescent secondary antibody staining

Cytokine/chemokine array. Six hours after CLP, mice were heavily anesthetized. Whole blood was drawn into ACD (˜1:9 volume) from the carotid artery. Plasma was isolated by centrifugation of blood at 4000 g for 10 minutes. Plasma was analyzed by Quansys Biosciences (Logan, Utah) to quantify cytokine and chemokine levels. Data are presented as mean±s.e.m. of six mice per condition. For H5N1 samples, six days after infection clarified mouse lung homogenates were prepared and inflammatory cytokine and chemokine profiles determined using mouse cytokine and chemokine arrays (Quansys Biosciences; Logan, Utah). Data are presented as mean±s.e.m. of three groups of pooled mice.

Lung virus titer determination. Performed as described in Sidwell, R. W., K. W. Bailey, M. H. Wong, D. L. Barnard, and D. F. Smee. 2005. In vitro and in vivo influenza virus-inhibitory effects of viramidine. Antiviral Res 68:10-17.

Lung development. Embryos were dissected, fixed, and rehydrated as described in Metzger, R. J., O. D. Klein, G. R. Martin, and M. A. Krasnow. 2008. The branching programme of mouse lung development. Nature 453:745-750. Lungs were serially immunostained with anti-PECAM (BD Pharmingen; clone MEC 13.3) and anti-E-cadherin (clone ECCD-2, Zymed) primary antibodies using a variation of the method described in Metzger et al.

Neutrophil migration. HL-60 cells were grown under standard conditions with RPMI-1640 media supplemented with 10% FBS and 1% pen/step. Cells induced with 1.2% dimethyl sulphoxide (DMSO) were obtained by seeding HL-60 cells at 3×10⁶ per mL in growth media and culturing for 4 6 days (Collins, S. J., F. W. Ruscetti, R. E. Gallagher, and R. C. Gallo. 1978. Terminal differentiation of human promyelocytic leukemia cells induced by dimethyl sulfoxide and other polar compounds. Proc Natl Acad Sci USA 75:2458-2462). hPMNs were isolated from healthy adult donor whole blood with ACD using techniques described in Zimmerman, G. A., T. M. McIntyre, and S. M. Prescott. 1985. Thrombin stimulates the adherence of neutrophils to human endothelial cells in vitro. J Clin Invest 76:2235-2246. The leukocyte chemoattractant fMLP (10 μM) along with Slit2 or Mock was placed in the lower wells of a 48-well chemotaxis chamber (Neuroprobe). A fibronectin coated (over night at 4° C.) polycarbonate membrane (Neuroprobe, 5 μm) was placed between the chemoattractant and the cells. HL-60 cells induced with DMSO or hPMNs (50 μl, 50,000 cells) were added to the upper wells. After incubating at 37° C. for 2 h, cells on the top surface of the filter were removed and cells that had migrated through the filter onto the undersurface were fixed and stained using Diff-Quic stain set (Dade Behring). Migrated cells in 5 high power fields were counted and migration expressed as the percent of cells migrated compared to cells migrated towards fMLP in the absence of Slit2 or Mock. Data are presented as s.e.m of at least three independent experiments.

Quantitative polymerase chain reaction (qPCR). Total RNA was extracted from the lungs of saline or LPS treated mice according to manufacturer s protocol (Nucleospin RNA II kit, Clontech). After reverse transcription, qPCR was performed with TaqMan assays (Applied Biosystems) for 18s rRNA and mouse Robo4. For hPMN studies, RNA was isolated using Trizol (Invitrogen) and qPCR was performed with TaqMan assays (Applied Biosystems) for human GAPDH and ROBO1-4.

Statistical analysis. The Student s t-test, log rank test, or ANOVA with post-hoc tests, where appropriate, were used to assess statistical significance. A P value of <0.05 was considered statistically significant.

It will be obvious to those having skill in the art that many changes may be made to the details of the above-described embodiments without departing from the underlying principles of the invention. The scope of the present invention should, therefore, be determined only by the following claims.

1. A method of promoting vascular barrier function in a subject, the method comprising:
administering to the subject a therapeutically effective amount of at least one Slit polypeptide, wherein administering the at least one Slit polypeptide results in the promotion of endothelial barrier function.

2. The method of claim 1, wherein the at least one Slit polypeptide is a ligand of Robo4.

3. The method of claim 1, wherein the at least one Slit polypeptide is at least one Slit2 polypeptide.

4. The method of claim 3, wherein the at least one Slit2 polypeptide is Slit2N (SEQ ID NO: 4).

5. The method of claim 1, wherein the at least one Slit polypeptide comprises the polypeptide sequence of at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and combinations, derivatives, homologs and analogs thereof.

6. The method of claim 1, wherein the promotion of vascular barrier function occurs in the presence of at least one mediator of inflammation selected from the group consisting of a lipopolysaccharide, TNF-α, IL-1β, and combinations thereof.

7. The method of claim 1, wherein the promotion of endothelial barrier function comprises at least one of promoting the presence of vascular endothelial cadherin (VE-cadherin) at the surface of vascular endothelial cells and promoting the expression of p120-catenin at the surface of vascular endothelial cells.

8. A method of promoting vascular barrier function in a subject, the method comprising:
administering to the subject a therapeutically effective amount of at least one inhibitor of at least one ARF GTP exchange factor (ARF-GEF), wherein modulating the at least one ARF-GEF results in the inhibition of vascular permeability in the subject.

9. The method of claim 8, wherein inhibiting the at least one ARF-GEF results in the inhibition of at least one ADP ribosylation factor (ARF).

10. The method of claim 9, wherein the at least one ARF is selected from the group consisting of ARF6, ARF1, and combinations thereof.

11. The method of any of claim 8, wherein the at least one inhibitor is a small molecule compound that inhibits at least one of the availability of the at least one ARF-GEF, the activation of the at least one ARF-GEF, and the activity of the at least one ARF-GEF.

12. The method of any of claim 8, wherein the at least one inhibitor of at least one ARF-GEF comprises SecinH3.

13. The method of any of claim 8, wherein the at least one inhibitor of at least one ARF-GEF comprises at least one of a compound according to Formula 1 and a compound according to Formula 2.

14. The method of any of claim 8, wherein the at least one inhibitor of at least one ARF-GEF is selected from one of: 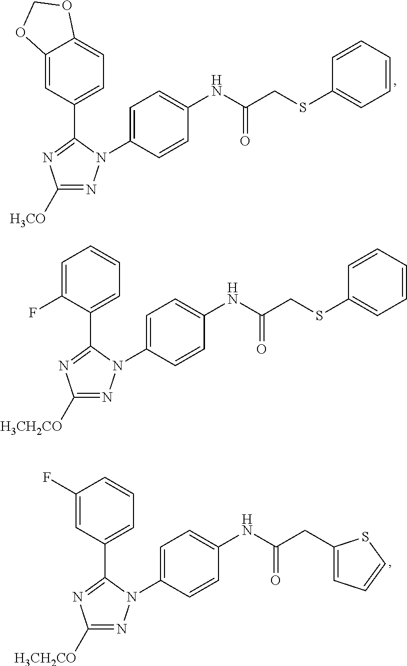
and pharmaceutically acceptable salts, solvates or hydrates thereof.

15. The method of claim 8, wherein the at least one inhibitor of at least one ARF-GEF inhibits a cytohesin selected from the ARNO family of cytohesins.

16. The method of claim 15, wherein the cytohesin is ARNO.

17. The method of claim 8, wherein the inhibition of vascular permeability in the subject occurs in the presence of at least one mediator of inflammation selected from the group consisting of a lipopolysaccharide, TNF-α, IL-1β, and combinations thereof.

18. The method of claim 8, wherein the inhibition of vascular permeability in the subject comprises at least one of promoting the presence of VE-cadherin at the surface of vascular endothelial cells and promoting the expression of p120-catenin at the surface of vascular endothelial cells.

19. A method of promoting the presence in a subject of VE-cadherin at the surface of vascular endothelial cells, the method comprising:
administering to the subject a therapeutic amount of at least one Slit polypeptide;
wherein administering to the subject at least one of a Slit polypeptide promotes the presence in the subject of VE-cadherin at the surface of vascular endothelial cells.

20. The method of claim 19, wherein the at least one Slit polypeptide is selected from at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and combinations, derivatives, homologs and analogs thereof.

21. The method of claim 19, wherein promoting the presence in the subject of VE-cadherin at the surface of vascular endothelial cells occurs in the presence of at least one mediator of inflammation selected from the group consisting of a lipopolysaccharide, TNF-α, IL-1β, and combinations thereof.

22. The method of claim 19, wherein administering the at least one Slit polypeptide promotes expression of p120-catenin at the surface of vascular endothelial cells.

23. A method of treating a subject with pulmonary vascular inflammation, the method comprising:
administering to the subject a therapeutically effective amount of at least one Slit polypeptide, wherein administering the at least one Slit polypeptide results in the reduction of pulmonary vascular inflammation in the subject.

24. The method of claim 23, wherein the at least one Slit polypeptide is at least one Slit2 polypeptide.

25. The method of claim 24, wherein the at least one Slit2 polypeptide is Slit2N (SEQ ID NO: 4).

26. The method of claim 23, wherein the at least one Slit polypeptide comprises the polypeptide sequence of at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and combinations, derivatives, homologs and analogs thereof.

27. The method of claim 23, wherein the reduction of vascular inflammation occurs in the presence of at least one mediator of inflammation selected from the group consisting of a lipopolysaccharide, TNF-α, IL-1β, and combinations thereof.

28. The method of claim 23, wherein the administering the at least one Slit polypeptide promotes the presence in the subject of VE-cadherin at the surface of vascular endothelial cells.

29. The method of claim 23, wherein the administering the at least one Slit polypeptide promotes the expression in the subject of p120-catenin at the surface of vascular endothelial cells.

30. The method of claim 23, wherein treating vascular inflammation comprises treating at least one of further comprising promoting in the subject the expression of p120-catenin at the surface of vascular endothelial cells.

31. A method of reducing vascular permeability associated with any of acute pulmonary vascular edema, chronic pulmonary vascular edema, acute pulmonary vascular inflammation, chronic pulmonary vascular inflammation, pulmonary fibrosis, including idiopathic pulmonary fibrosis, bacterial sepsis, or influenza infection, the method comprising:
administering to the subject a therapeutically effective amount of at least one Slit polypeptide, wherein administering the at least one Slit polypeptide reduces vascular permeability associated with a pathological condition in the subject.

32. The method of claim 31, wherein the pathological condition is pulmonary fibrosis, including idiopathic pulmonary fibrosis.

33. The method of claim 31, wherein the at least one Slit polypeptide is administered to the subject in the presence of at least one mediator of inflammation selected from the group consisting of a lipopolysaccharide, TNF-α, IL-1β, and combinations thereof.

34. The method of claim 31, wherein the administering at least one Slit polypeptide promotes the presence of VE-cadherin at the surface of vascular endothelial cells.

35. A method of treating a subject suffering from pulmonary fibrosis, the method comprising:
administering to the subject a therapeutically effective amount of a compound selected from a compounds according to Formula 1 and a compound according to Formula 2.

36. The method of claim 35, comprising administering to the subject a therapeutically effective amount of SecinH3 and pharmaceutically acceptable salts, solvates or hydrates thereof.

37. The method of claim 35, comprising administering to the subject a therapeutically effective amount of a compound selected from one of: 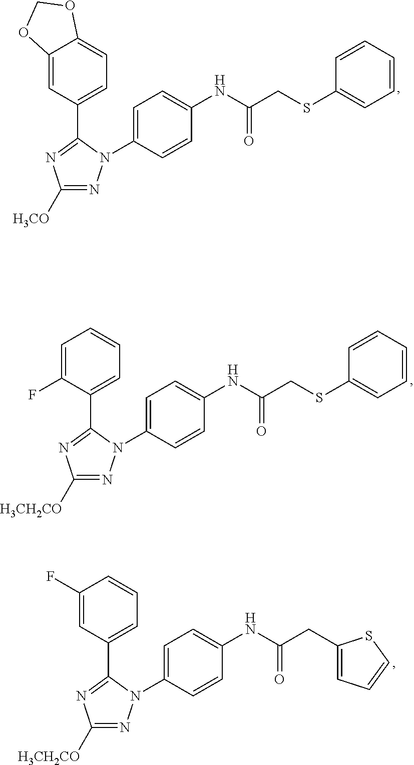
and pharmaceutically acceptable salts, solvates or hydrates thereof.

38. A method of inhibiting the occurrence of pulmonary fibrosis in a subject, the method comprising:
administering to the subject a therapeutically effective amount of a compound selected from a compounds according to Formula 1 and a compound according to Formula 2.

39. The method of claim 38, comprising administering to the subject a therapeutically effective amount of SecinH3 and pharmaceutically acceptable salts, solvates or hydrates thereof.

40. The method of claim 38, comprising administering to the subject a therapeutically effective amount of a compound selected from one of: 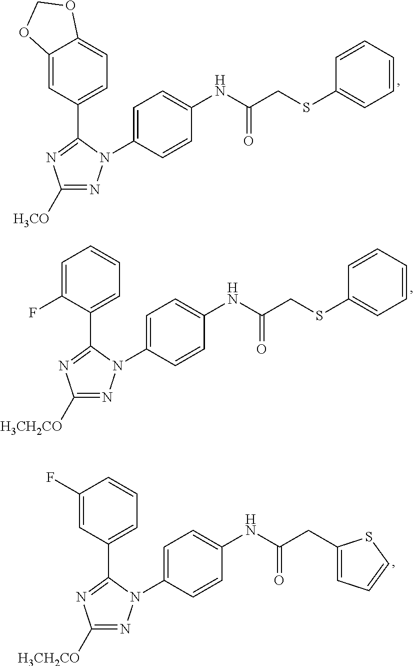
and pharmaceutically acceptable salts, solvates or hydrates thereof.

41. A method of treating a subject suffering from pulmonary fibrosis, the method comprising:
administering to the subject a therapeutically effective amount of a Slit polypeptide.

42. The method of claim 41, comprising administering to the subject a therapeutically effective amount of a Slit2 polypeptide.

43. The method of claim 42, wherein the Slit2 polypeptide is Slit2N (SEQ ID NO: 4).

44. The method of claim 41, wherein the Slit polypeptide is selected from one of the polypeptides represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and combinations, derivatives, homologs and analogs thereof.

45. A method of inhibiting the occurrence of pulmonary fibrosis in a subject, the method comprising:
administering to the subject a therapeutically effective amount of a Slit polypeptide.

46. The method of claim 45, comprising administering to the subject a therapeutically effective amount of a Slit2 polypeptide.

47. The method of claim 45, wherein the Slit polypeptide is selected from one of the polypeptides represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and combinations, derivatives, homologs and analogs thereof.
